(19) United States
(12) Patent Application Publication
(10) Pub. No.: US 2014/0094588 A1
(43) Pub. Date: Apr. 3, 2014

(54) PROGNOSTIC AND THERAPEUTIC SIGNATURE FOR MALIGNANT MELANOMA

(71) Applicants: Stefanie Meyer, Regensburg (DE); Anja Bosserhoff, Regensburg (DE); Peter Wild, Geiselhoring (DE); Thomas Fuchs, Graz (AT)

(72) Inventors: Stefanie Meyer, Regensburg (DE); Anja Bosserhoff, Regensburg (DE); Peter Wild, Geiselhoring (DE); Thomas Fuchs, Graz (AT)

(73) Assignees: Universität Regensburg, Regensburg (DE); Stefanie Meyer, Regensburg (DE)

(21) Appl. No.: 14/008,222

(22) PCT Filed: Mar. 30, 2012
(86) PCT No.: PCT/EP2012/055827

§ 371 Date: Dec. 9, 2013

(30) Foreign Application Priority Data
Apr. 1, 2011 (EP) 11002747.1

(51) Int. Cl. G01N 33/574

(52) U.S. Cl. CPC G01N 33/5743
USPC 530/351; 436/501; 435/25; 435/15; 506/9

(57) ABSTRACT

The present invention relates to a method of predicting the course of disease in a patient having a malignant melanoma, the method comprising determining in melanoma cells comprised in a sample obtained from said malignant melanoma the presence or amount of at least five biomarkers selected from the group comprising or consisting of MTAP, PTEN, Bax, Bcl-X, β-Catenin, CD20, Cox-2, CD49d and MLH1, wherein the absence or decreased amount of MTAP and β-Catenin and/or the presence or increased amount of PTEN, Bax, Bcl-X, CD20, Cox-2, CD49d and MLH1, is associated with a disadvantageous course of disease.

The present invention relates to a method of predicting the course of disease in a patient having a malignant melanoma, the method comprising determining in melanoma cells comprised in a sample obtained from said malignant melanoma the presence or amount of at least five biomarkers selected from the group comprising or consisting of MTAP, PTEN, Bax, Bcl-X, β-Catenin, CD20, Cox-2, CD49d and MLH1, wherein the absence or decreased amount of MTAP and β-Catenin and/or the presence or increased amount of PTEN, Bax, Bcl-X, CD20, Cox-2, CD49d and MLH1 is associated with a disadvantageous course of disease. The present invention further relates to a method of preparing a tailored pharmaceutical composition for a patient having a malignant melanoma, a kit for predicting the course of disease in a patient having a malignant melanoma, a kit for preparing a tailored pharmaceutical composition for a patient having a malignant melanoma as well as a pharmaceutical composition for use in treating or preventing malignant melanoma.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Cutaneous malignant melanoma (MM), represents the most common cause of death from skin cancer, and, apart from female lung cancer, it is the tumour entity with the highest increase of incidence worldwide (Jemal, A et al. 2008). Malignant melanoma is characterized by a multi-factorial aetiology. Sun exposure and genetic susceptibility have been proposed as major aetiological and predisposing factors and may explain the reported increase of incidence to some degree (Lens, M. B. et al 2004.)

The metastatic stage IV of malignant melanoma with an average 10-year survival rate ranging from 9% to 15% (depending on its pattern of metastasis; Balch, C. M. et al., 2001) cannot yet be cured and improvement in overall survival among these patients remains an elusive goal. Despite novel therapeutic approaches, the prognosis of patients suffering from metastatic stage IV malignant melanoma remains unfavourable (Agarwala, S. S. 2009.)

De facto, the prognosis of patients with malignant melanoma may only in part be derived from clinical and histological parameters. According to the AJCC 2009 classification Balch, C. M. et al 2009,) the findings of vertical tumour thickness (Breslow, A. et al., 1970,) tumour ulceration (Grande Sarpa H. et al., 2006) and sentinel node biopsy (Morton, D. L. et al., 2006) represent the most dominant prognostic factors. In stage pT1 melanomas (≦1.00 thickness), the mitotic rate (histologically defined as mitoses/mm²) has to be considered as additional prognostic parameter (Balch, C. M. et al 2009). These current staging methods such as tumour thickness, ulceration and invasion of the sentinel node are known to be prognostic parameters in patients with malignant melanoma. However, predictive molecular marker profiles for risk stratification and therapy optimization are not yet available for routine clinical assessment.

Rothberg et al. (Gould Rothberg, B. E. et al., 2009) describe a meta-analysis of published literature to identify associations between immunohistochemical expression and survival outcomes in melanoma. Promising markers identified by Rothberg and co-workers include MUC18, MMP-2, Ki-67, PCNA and P16/INK4A. The authors conclude that these results require validation in adequately powered studies.

Rothberg and Rimm (Gould Rothberg, B. E. et al., 2010) describe the analysis of data not eligible for the meta-analysis performed in Rothberg et al. (Gould Rothberg, B. E. et al., 2009) but nonetheless of potential value in providing a prioritised list of protein candidates for further studies with the aim of identifying prospective prognostic markers. The authors provide a list of proteins that they recommend as a priority set for inclusion in studies of melanoma prognosis.

Alonso et al. (Alonso, S. R. et al., 2004) describe protein expression profiles at the different stages of malignant melanoma progression. A predictor model for survival was established, including the proteins p16^{INK4a}, Ki67, P21^{CIP1} and Bcl-6. The proteins BCLX-L, MLH-1 and TOP2A, although analyzed, were not further considered in the prognostic model.

Wild et al. (Wild, P. J. et al., 2006) describe a reduced expression of MTAP in primary malignant melanomas and in melanoma metastases compared with benign nevi. However, in the overall cohort, MTAP expression was not associated with prognosis. Instead, MTAP expression was found to correlate with responsiveness to interferon therapy.

In a later study based on a larger cohort of patients, Meyer et al. (Meyer, S. et al., 2010) further investigated whether expression of MTAP is of prognostic or therapeutic relevance in patients with melanoma. An association between MTAP immunoreactivity and overall survival as well as recurrence-free survival was shown in this patient group.

Meyer et al. (Meyer, S. et al., 2009) describe a study to correlate Cox-2 immunoreactivity in tumours to the outcome of patients with malignant melanoma. Cox-2 expression was found to be significantly increased from nevi to primary malignant melanoma and metastases and Cox-2 positivity was associated with shorter recurrences-free survival of the patients. The authors concluded that Cox-2 expression in primary malignant melanoma indicates an increased risk of tumour recurrence. However, no association with longer progression-free survival could be shown in patients with malignant melanoma metastases who had received biomodulatory therapy. Thus, the authors conclude that Cox-2 might mainly contribute to early steps in melanoma progression, such as growth and invasion of primary malignant melanoma but might be less essential in the advanced metastatic setting of melanoma disease. Nonetheless, a different study by (Reichle, A. et al., 2007) describes a phase II trial showing beneficial effects of a Cox-2 inhibitor in patients with stage IV (i.e. metastatic) melanoma.

WO 2008/141275 describes a large amount of molecular markers expressed at certain stages of malignant melanoma and states that said markers may be employed to predict the malignancy potential of a malignant melanoma and to determine the correct treatment regimen. However, no correlation of molecular markers with the course of disease, such as recurrence-free survival or overall survival, is shown in WO 2008/141275.

Despite the fact that hundreds of studies sought to assess the potential prognostic value of molecular markers in predicting the course of cutaneous malignant melanoma, according to the latest review meta-analyzes (Gould Rothberg, B. E. et al., 2009; Gould Rothberg, B. E. et al., 2010) there are no predictive molecular profiles for risk association or therapy optimisation applicable for routine clinical assessment of malignant melanoma. Furthermore, the variability of clinical behaviour in patients with malignant melanoma can only partially be explained by clinical and histological data and, thus, there is a need to identify biological marker profiles for use in assigning patients to a specific risk group.

This need is addressed by the provision of the embodiments characterised in the claims.

Accordingly, the present invention relates to a method of predicting the course of disease in a patient having a malignant melanoma, the method comprising determining in melanoma cells comprised in a sample obtained from said malignant melanoma the presence or amount of at least five biomarkers selected from the group comprising or consisting of MTAP, PTEN, Bax, Bcl-X, β-Catenin, CD20, Cox-2, CD49d and MLH1, wherein the absence or decreased amount of MTAP and β-Catenin and/or the presence or increased amount of PTEN, Bax, Bcl-X, CD20, Cox-2, CD49d and MLH1 is associated with a disadvantageous course of disease.

In accordance with the present invention, the term “predicting the course of disease” refers to the provision of an assessment whether the malignant melanoma has a favourable progression/outcome or an unfavourable progression/outcome. The term disease, in this regard, refers to malignant melanoma. A favourable progression/outcome, also referred to herein as an advantageous course of disease, relates to no risk or a low risk of recurrence of malignant melanoma and/or a long time of disease-free survival, such as for example more than 5 years. An unfavourable progression/outcome, also referred to herein as a disadvantageous course of disease, relates to a high risk of recurrence of malignant melanoma, including e.g. the formation of local malignant melanoma as well as the formation of regional or distant metastases, and/or a short time of disease-free survival such as e.g. less than 5 years and/or a short overall survival time. The term “recurrence”, as used herein, relates to the repeated outbreak of malignant melanoma, or a progression of the malignant melanoma such as for example in terms of formation of metastases from the malignant melanoma analyzed but independently of whether the disease was cured before said outbreak or progression.

As used herein, the term “malignant melanoma” refers to a type of skin cancer well known in the medical art. Melanoma is the type of skin cancer that has the highest grade of malignancy. Among cells composing skin, melanin-pigment-producing cells are referred to as pigment cells or melanocytes. When these melanocytes become cancerous, a malignant melanoma is developed. Malignant melanoma is staged according to the severity of the disease into stage 0 to stage 4. Stage 0 refers to melanoma in situ with 99.9% survival. Stage I/II refers to invasive melanoma with 85 to 99% survival and is further divided into T1a (less than 1.00 mm primary tumour thickness, without ulceration and mitosis<1/mm²), T1b (less than 1.00 mm primary tumour thickness, with ulceration or mitoses≧1/mm²) and T2a (1.00 to 2.00 mm primary tumour thickness, without ulceration). Stage II refers to high risk melanoma with 40 to 85% survival and is further divided into T2b (1.00 to 2.00 mm primary tumour thickness, with ulceration), T3a (2.00 to 4.00 mm primary tumour thickness, without ulceration), T3b (2.00 to 4.00 mm primary tumour thickness, with ulceration), T4a (4.00 mm or greater primary tumour thickness without ulceration) and T4b (4.00 mm or greater primary tumour thickness with ulceration). Stage III refers to regional metastasis with 25 to 60% survival and is further divided into N1 (single positive lymph node), N2 (2 to 3 positive lymph nodes or regional skin/in-transit metastasis) and N3 (four positive lymph nodes or lymph node and regional skin/in-transit metastases). Finally, stage IV refers to distant metastasis with only 9 to 15% survival. Stage IV is further divided into M1a (distant skin metastasis and normal lactate dehydrogenase), M1b (lung metastasis, normal lactate dehydrogenase) and M1c (other distant metastasis or any distant metastasis with elevated lactate dehydrogenase).

The term “melanoma cells”, as used herein, refers to melanocytes that have become cancerous. Melanocytes, including melanoma cells, are well known to the skilled person and can be easily identified in a sample due to their location in the stratum basale of the epidermis as well as via melanocyte-specific markers including, but not limited to, Melan-A, HMB45, Protein S100, DCT and TRP2.

The term “biomarker selected from the group [ . . . ]” according to the present invention relates to the recited markers in any of their naturally occurring forms, including nucleic acid molecules such as e.g. DNA, including cDNA or genomic DNA, and RNA as well as proteins.

As used herein, the term “determining the [ . . . ] presence” refers to determining whether the analyzed biomarker is present or absent in melanoma cells comprised in the sample investigated. The biomarker is considered present in accordance with the present invention when it is detected in amounts exceeding the standard procedural error, such as for example observed in the form of background staining obtained in immunohistochemical or western blot analyzes. The skilled person knows how to determine such procedural errors, for example by analyzing non-disease control samples or by omitting certain steps or compounds in the procedure, such as for example a primary antibody in immunohistochemical stainings or a template in nucleic acid amplification techniques etc. In the case that the amount of biomarker detected corresponds to or is less than the standard procedural error, e.g. the background staining in an immunohistochemical analysis, the biomarker is considered as not being present in the sample.

As used herein, the term “determining the [ . . . ] amount” refers to quantitatively or semi-quantitatively determining amounts of the respective biomarkers. Quantitative analysis refers to the determination of absolute or normalized (e.g. compared to a non-changing reference marker) values of biomarker amounts, such as e.g. copy numbers of nucleic acids or intensities of stainings in immunohistochemical or western blot techniques. Furthermore, the number of stained cells versus cells not stained may be evaluated. Semi-quantitative analysis refers to the determination of relative amounts, such as for example by visual analysis of immunohistochemically stained samples by a skilled person, such as e.g. a dermato-histopathologist or a surgical pathologist. As described in the appended examples, such analyzes may be performed based on a step-wise scoring system allocating different scores to samples containing e.g. no staining, weak staining, moderate staining, strong staining, very strong staining etc.

The term “decreased amount”, as used herein, refers to lower expression levels of the biomarker of interest in melanoma cells in the sample obtained from the malignant melanoma as compared to expression levels observed in a control sample, such as for example non-malignant tissues. Preferably, the term relates to statistically significant lower expression levels of the biomarker of interest in melanoma cells in the sample obtained from the malignant melanoma of interest as compared to expression levels observed in the control tissues. Expression levels observed in non-malignant tissues may for example be analyzed in a parallel control experiment based on a disease-free sample, such as for example on benign nevi obtained from the same patient. The amount of biomarker is considered to be decreased when its amount is at least 10% lower in the malignant melanoma sample than in non-malignant tissues, such as for example at least 20% lower, at least 30% lower, at least 40% lower, at least 50% lower, at least 75% lower, at least 100% lower (i.e. twice as low), at least 200% lower, at least 300% lower, at least 500% lower etc.

The term “increased amount”, as used herein, refers to higher expression levels of the biomarker of interest in melanoma cells in the sample obtained from the malignant melanoma as compared to expression levels observed in a control sample, such as for example non-malignant tissues. Preferably, the term relates to statistically significant higher expression levels of the biomarker of interest in melanoma cells in the sample obtained from the malignant melanoma of interest as compared to expression levels observed in the control tissues. Expression levels observed in non-malignant tissues may for example be analyzed in a parallel control experiment based on a disease-free sample, such as for example on benign nevi obtained from the same patient. The amount of biomarker is considered to be abnormally increased when its amount is at least 10% higher in the malignant melanoma sample than in non-malignant tissues, such as for example at least 20% higher, at least 30% higher, at least 40% higher, at least 50% higher, at least 75% higher, at least 100% higher (i.e. twice as high), at least 200% higher, at least 300% higher, at least 500% higher etc.

The term “expression level”, as used herein, refers to a value of expression of a particular marker in a sample of interest. The expression level of a marker corresponds to the number of copies of the expression product of the corresponding gene, either on a nucleic acid level (e.g. mRNA) or on the protein level. Thus, the determination of the expression level of a particular marker can, for example, be carried out on the nucleic acid level or on the level of the respective protein encoded by said gene.

Methods for the determination of expression levels of a protein on the amino acid level include but are not limited to immunohistochemical methods as described in the appended examples but also e.g. Western blotting or polyacrylamide gel electrophoresis in conjunction with protein staining techniques such as Coomassie Brilliant blue or silver-staining. For these latter methods, the total protein is loaded onto a polyacrylamide gel and separated by electrophoresis. Afterwards, the separated proteins are transferred onto a membrane, e.g. a polyvinyldifluoride (PVDF) membrane, by applying an electrical current. The proteins on the membrane are exposed to an antibody specifically recognizing the protein of interest. After washing, typically a second antibody specifically recognizing the first antibody and carrying a readout system such as a fluorescent dye is applied. The amount of the protein of interest is often determined by comparing the fluorescence intensity of the protein derived from a sample of the patient of interest with the fluorescence intensity obtained with the protein derived from a control sample. Also of use in protein quantification is the Agilent Bioanalyzer technique.

Methods for determining expression levels on the nucleic acid level include, but are not limited to, Northern blotting, PCR, RT-PCR or real RT-PCR. PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and terminates all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed, for example, in a 50 μl reaction mixture containing 5 μl of 10×PCR buffer with 1.5 mM MgCl₂, 200 μM of each deoxynucleoside triphosphate, 0.5 μl of each primer (10 μM), about 10 to 100 ng of template DNA and 1 to 2.5 units of Taq Polymerase. The primers for the amplification may be labelled or be unlabelled. DNA amplification can be performed, e.g., with a model 2400 thermal cycler (Applied Biosystems, Foster City, Calif.): 2 min at 94° C., followed by 30 to 40 cycles consisting of annealing (e. g. 30 s at 50° C.), extension (e. g. 1 min at 72° C., depending on the length of DNA template and the enzyme used), denaturing (e. g. 10 s at 94° C.) and a final annealing step at 55° C. for 1 min as well as a final extension step at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus Vent, Amplitaq, Pfu and KOD, some of which may exhibit proof-reading function and/or different temperature optima. The person skilled in the art knows how to optimize PCR conditions for the amplification of specific nucleic acid molecules with primers of different length and/or composition or to scale down or increase the volume of the reaction mix. The “reverse transcriptase polymerase chain reaction” (RT-PCR) is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerizing agents that convert the RNA target sequence into a complementary, copy-DNA (cDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746,121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT Reaction can be performed, for example, in a 20 μl reaction mix containing: 4 μl of 5×AMV-RT buffer, 2 μl of Oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70 C.° for 15 minutes to allow for reverse transcription. The reaction temperature is then raised to 95 C.° for 1 minute to denature the RNA-cDNA duplex. Next, the reaction temperature undergoes two cycles of 95° C. for 15 seconds and 60 C.° for 20 seconds followed by 38 cycles of 90 C.° for 15 seconds and 60 C.° for 20 seconds. Finally, the reaction temperature is held at 60 C.° for 4 minutes for the final extension step, cooled to 15 C.°, and held at that temperature until further processing of the amplified sample. Any of the above mentioned reaction conditions may be scaled up according to the needs of the particular case.

The resulting products may be loaded onto an agarose gel and band intensities are compared after staining the nucleic acid molecules with an intercalating dye such as ethidiumbromide or SybrGreen.

Real-time PCR employs a specific probe, in the art also referred to as TaqMan probe, which has a reporter dye covalently attached at the 5′ end and a quencher at the 3′ end. After the TaqMan probe has been hybridized in the annealing step of the PCR reaction to the complementary site of the polynucleotide being amplified, the 5′ fluorophore is cleaved by the 5′ nuclease activity of Taq polymerase in the extension phase of the PCR reaction. This enhances the fluorescence of the 5′ donor, which was formerly quenched due to the close proximity to the 3′ acceptor in the TaqMan probe sequence. Thereby, the process of amplification can be monitored directly and in real time, which permits a significantly more precise determination of expression levels than conventional end-point PCR. Also of use in Real-time RT-PCR experiments is a DNA intercalating dye such as SybrGreen for monitoring the de novo synthesis of double stranded DNA molecules.

The skilled person is aware that mutations and/or variations in the genes encoding the biomarkers in accordance with the present invention as well as in the regulatory elements of said genes (e.g. promoters, enhancers etc.) may be causative or associated with a change of expression levels of said biomarkers. For example, PTEN mutations and deficiencies are prevalent in many types of human cancers, leading to a loss of functional PTEN protein in those cancers (Mirmohammadsadegh et al. 2006; Lahtz et al. 2010; Zhou et al. 2000; Zhang and Yu 2010). Furthermore, promoter hyper-methylation has been shown to lead to a loss of MTAP expression (Behrmann et al. 2003). Thus, it is also envisaged herein that the determination of the presence or amount of the biomarkers in accordance with the present invention is based on the detection of mutations (i.e. genetic changes) or variations (i.e. epigenetic changes) of said biomarkers. Methods for determining mutations and/or variations in genes are well known in the art and include, without being limiting PCR based techniques, DNA sequencing-based techniques, hybridization-based techniques, single-strand conformation polymorphism analysis (SSCA), denaturating gradient gel electrophoresis (DGGE), mismatch cleavage detection, heteroduplex analysis, primer extension-based techniques, 5′-nuclease assay-based techniques, using antibodies or sequence-specific DNA-binding proteins as well as methylation-sensitive arbitrarily primed PCR (e.g. Gonzalgo et al. 1997, Cancer Res. 57:594-599), quantitative methylation-specific PCR (Q-MSP; as described e.g. in Current Protocols in Human Genetics, DOI: 10.1002/0471142905.hg1006s61), methylation-sensitive restriction analysis (e.g. Singer-Sam et al. 1990, Nucl. Acids Res. 18:687), methylation-quantification of endonuclease-resistant DNA (Bettstetter et al. 2008) or methylation-sensitive sequencing methods (such as e.g. bisulfite DNA sequencing; Frommer et al. 1992, PNAS 89:1827-1831).

Said techniques are well known to the person skilled in the art.

Non-limiting examples for nucleic acid amplification assays and means to perform such include PCR, (including nested PCR, RT-PCR, quantitative real-time detection, PCR extension assays, Nucleic Acid Sequence Base Amplification (NASBA), single-strand confirmation polymorphism (SSCP) PCR, PCR-restriction enzyme fragment length polymorphism (RFLP) analysis), amplification refractory mutation systems (ARMS™) and amplification refractory mutation system linear extension (ALEX™) assays. Details of such methods can be found in art, see, for example, Newton et al., Nucleic Acids Res. 17 (1989) 2503-2516; Agrawal (Ed.), “Protocols for Oligonucleotides and Analogs: Synthesis and Properties (Methods in Molecular Biology, 20)”, Humana Press, 1993; Hague et al., Diagn. Mol. Pathol. 7 (1998) 248-252; Innis et al. (Ed.), “PCR Applications: Protocols for Functional Genomics”, Academic Press, 1999; Chen and Janes (Ed.), “PCR Cloning Protocols: From Molecular Cloning to Genetic”, 2nd edition, Humana Press, 2002; Pissard et al., Clin. Chem. 48 (2002) 769-772; Blondal et al., Nucleic Acids Res 31 (2003) e155; Steemers et al., Nature Meth. 3 (2006) 31-33; Kakavas et al., J. Clin. Lab. Anal. 20 (2006) 1-7.

Examples for sequencing assays comprise without limitation approaches of sequence analysis by direct sequencing, fluorescent SSCP in an automated DNA sequencer and Pyrosequencing. These procedures are common in the art, see e.g. Adams et al. (Ed.), “Automated DNA Sequencing and Analysis”, Academic Press, 1994; Alphey, “DNA Sequencing: From Experimental Methods to Bioinformatics”, Springer Verlag Publishing, 1997; Ramon et al., J. Transl. Med. 1 (2003) 9; Meng et al., J. Clin. Endocrinol. Metab. 90 (2005) 3419-3422.

Examples for hybridization assays comprise without limitation Northern and Southern blot assays, heteroduplex analysis, detection of mutations by sequence specific oligonucleotide hybridization, allele-specific oligonucleotide hybridization on DNA chips, assays based on IIlumina's® technology, assays based on the BeadArray® technology, see, for example, Barnes et al., Nucleic Acids Res. 33 (2005) 5914-5923; Fan et al., Biotechniques 39 (2005) 583-588; Shen et al., Mutat. Res.—Fund. Mol. M. 573 (2005) 70-82; Steemers and Gunderson, Pharmacogenomics, 6 (2005) 777-782.

The term “at least five biomarkers”, as used herein, refers to five or more biomarkers. Preferably, said term relates to at least six biomarkers, more preferably at least seven biomarkers. The term also relates to at least eight biomarkers or at least nine biomarkers. The term further encompasses exactly five or exactly six or exactly seven or exactly eight or exactly nine biomarkers. In accordance with this method of the invention, the presence or amount of at least five biomarkers selected from the recited list is determined. Also encompassed by the method is that additional biomarkers and/or reference markers are analyzed, wherein said additional biomarkers may or may not be selected from the recited list of biomarkers. In other words, whereas the at least five biomarkers have to be chosen from MTAP, PTEN, Bax, Bcl-X, β-Catenin, CD20, Cox-2, CD49d and MLH1, further different biomarkers and/or reference markers may additionally be analyzed in accordance with the present invention.

The term “reference marker”, as used herein, refers to a marker that is present in melanocytes at substantially constant levels. In other words, the expression level of a reference marker should not differ (apart from deviations caused by the limits of accuracy of established detection methods) between samples of different origin and between benign and malignant samples. Due to the essentially unchanged amounts of such reference markers in different samples, they may be employed to normalise values of biomarker amounts, e.g. by comparison between the expression levels of said non-changing reference marker with the expression level of the biomarker of interest. Often, housekeeping genes are used as reference markers. Examples of reference markers include, without being limiting, GAPDH, RPLP0, PGK1, HSP90AB1, cyclophilin, actin and many more. Further examples are detailed for example in Eisenberg et al. 2003 (Trends Genet 19:362-5) and Velculescu et al. 1999 (Nat Genet 23:387-8.).

In accordance with the present invention, the term “normalizing values of biomarker amounts” or “normalizing the expression levels” relates to a correction of the measured value. This correction is usually carried out in order to control for bias introduced during the process of sample collection and analysis, which can for example arise due to variations based on different laboratories and/or different machines used, due to differences in staining protocols or differences between extraction protocols that may co-purify inhibitors, and due to different reverse transcription and PCR efficiencies. Importantly, normalisation enables a direct comparison of values obtained from individual patients and/or in different laboratories.

Several strategies for normalisation are known in the art. For example, in case of immunohistochemical methods or quantitative PCR measurement, normalizing may be carried out against the expression level(s) of (an) internal reference gene(s)/reference protein(s), which is determined in the same sample; against sample size; against total amount of RNA or genomic DNA or protein; or against an artificially introduced molecule of known amount.

Normalisation is preferably achieved by mathematically dividing the expression values from the marker to be investigated by the expression values of a reference marker. This is particularly preferred if the expression values are given in a linear scale. If the expression values are expressed in a logarithmic scale, normalisation is achieved by subtracting the expression value of the reference marker from the expression value of the marker of interest.

In case of microarray analysis normalisation techniques including, without being limiting, RMA, GCRMA, MASS, dChip and VSN and others could be used to process raw data to achieve comparability. Details of these methods can be found in Stafford (2008) “Methods in microarray Normalisation” (ISBN-13: 978-1420052787) and Quakenbush Nat. Gene. 2002 (Nat Genet; 32 Suppl:496-501)).

In accordance with the present invention, the term “MTAP” refers to S-methyl-5′-thioadenosine phosphorylase, which plays a major role in polyamine metabolism and is important for the salvage of both adenine and methionine. MTAP protein is characterised by the EC number 2.4.2.28. Human MTAP is for example represented by the Entrez Gene ID 4507 and UniProt ID Q13126 and is shown for example in SEQ ID NOs: 1 and 2. MTAP has been described in the art, for example in Behrmann et al., 2003.

As used herein, the term “PTEN” refers to the phosphatidylinositol-3,4,5-trisphosphate 3-phosphatase and dual-specificity protein phosphatase, which plays a major role as a tumour suppressor. PTEN acts as a dual-specificity protein phosphatase, dephosphorylating tyrosine-, serine- and threonine-phosphorylated proteins. PTEN also acts as a lipid phosphatase, removing the phosphate in the D3 position of the inositol ring from phosphatidylinositol 3,4,5-trisphosphate, phosphatidylinositol 3,4-diphosphate, phosphatidylinositol 3-phosphate and inositol 1,3,4,5-tetrakisphosphate. PTEN protein is characterised by the EC numbers 3.1.3.16, 3.1.3.48 and 3.1.3.67. Human PTEN is for example represented by the Entrez Gene ID 5728 and UniProt ID P60484 and is shown for example in SEQ ID NOs: 3 and 4 PTEN has been described in the art, for example in Zhang and Yu 2010.

The term “Bax”, as used herein refers to the BCL2-associated X protein, which accelerates programmed cell death by binding to, and antagonising the apoptosis repressor BCL2 or its adenovirus homolog E1B 19k protein. Bax also induces the release of cytochrome c, activation of CASP3, and thereby apoptosis. Human Bax is for example represented by the Entrez Gene ID 581 and UniProt ID Q07812 and is shown for example in SEQ ID NOs: 5 and 6. Bax has been described in the art, for example in Lowe et al. 2004.

As used herein, the term “Bcl-X” refers to the Bcl-2-like protein 1, which is a potent inhibitor of cell death and inhibits the activation of caspases. Human Bcl-X is for example represented by the Entrez Gene ID 598 and UniProt ID Q07817 and is shown for example in SEQ ID NOs: 7 and 8. Bcl-X has been described in the art, for example in Lowe et al. 2004.

As used herein, the term “β-Catenin” refers to CTNNB1, which is involved in the regulation of cell adhesion. The majority of β-catenin is localized to the cell membrane and is part of E-cadherin/catenin adhesion complexes which are proposed to couple cadherins to the actin cytoskeleton. It is also involved in signal transduction through the Wnt pathway and nuclear β-catenin is involved in transcriptional regulation by association with transcription factors of the TCF/LEF family. Human β-Catenin is for example represented by the Entrez Gene ID 1499 and UniProt ID P35222 and is shown for example in SEQ ID NOs: 9 and 10. β-Catenin has been described in the art, for example in Delmas et al. 2007.

The term “CD20”, as used herein refers to the B-lymphocyte cell-surface antigen B1, also having the gene name MS4A1. CD20 is considered to play an important role in the regulation of B-cell activation and proliferation. Human CD20 is for example represented by the Entrez Gene ID 931 and UniProt ID P11836 and is shown for example in SEQ ID NOs: 11 and 12. CD20 has been described in the art, for example in Zabierowski and Herlyn 2008.

In accordance with the present invention, the term “Cox-2” refers to the prostaglandin-endoperoxide synthase 2, also referred to as PTGS2. Cox-2 mediates the formation of prostaglandins from arachidonate and may also have a role as a major mediator of inflammation and/or a role for prostanoid signaling in activity-dependent plasticity. The Cox-2 protein is characterised by the EC number 1.14.99.1. Human CD20 is for example represented by the Entrez Gene ID 5743 and UniProt ID P35354 and is shown for example in SEQ ID NOs: 13 and 14. Cox-2 has been described in the art, for example in Meyer et al. 2009.

As used herein, the term “CD49d” refers to integrin alpha 4, also referred to as ITGA4, antigen CD49D or alpha 4 subunit of VLA-4 receptor. Integrin alpha-4/beta-1 (VLA-4) and alpha-4/beta-7 are receptors for fibronectin. They recognise one or more domains within the alternatively spliced CS-1 and CS-5 regions of fibronectin. They are also receptors for VCAM1. Integrin alpha-4/beta-1 recognises the sequence Q-I-D-S in VCAM1. Integrin alpha-4/beta-7 is also a receptor for MADCAM1. It recognises the sequence L-D-T in MADCAM1. On activated endothelial cells integrin VLA-4 triggers homotypic aggregation for most VLA-4-positive leukocyte cell lines. It may also participate in cytolytic T-cell interactions with target cells. Human CD49d is for example represented by the Entrez Gene ID 3676 and UniProt ID P13612 and is shown for example in SEQ ID NOs: 15 and 16. CD49d has been described in the art, for example in Kuphal et al. 2005.

The term “MLH1”, as used herein refers to the DNA mismatch repair protein Mlh1, which heterodimerises with PMS2 to form MutL alpha, a component of the post-replicative DNA mismatch repair system. It also heterodimerises with MLH3 to form MutL γ, which plays a role in meiosis and has further been implicated in DNA damage signaling, a process which induces cell cycle arrest and can lead to apoptosis in case of major DNA damages. Human MLH1 is for example represented by the Entrez Gene ID 4292 and UniProt ID P40692 and is shown for example in SEQ ID NOs: 17 and 18. MLH1 has been described in the art, for example in Korabiowska et al. 2006.

In accordance with this method of the present invention, the absence or decreased amount of MTAP and/or β-Catenin is associated with an disadvantageous course of the disease, i.e. the malignant melanoma. In other words, when one or both of these markers are found to be absent or lowered in melanoma cells comprised in a sample obtained from a patient, then this increases the likelihood of said patient to have a recurrence of the disease. The presence of one or both of these markers or elevated levels thereof, however, render it more likely that the patient will not have a recurrence of the disease. Furthermore, in accordance with this method of the invention, the presence or increased amount of PTEN, Bax, Bcl-X, CD20, Cox-2, CD49d and/or MLH1 is associated with a disadvantageous course of disease. Thus, when one or more of these markers are found to be expressed or elevated in melanoma cells comprised in a sample obtained from a patient, then this renders it more likely that the patient will have a recurrence of the disease while the absence of one or more of these markers or decreased levels thereof increase the likelihood of said patient to not have a recurrence of the disease.

In accordance with the present invention, it was found that a set of 9 specific markers enables the characterisation of melanoma cells comprised in a tissue sample of a malignant melanoma, thereby providing an independent prediction model for clinical outcome and individualized, targeted therapy options in patients with malignant melanoma. A prognostic score based on the expression levels of said limited set of markers achieved a higher prognostic accuracy than any other previously used combination of prognostic markers in malignant melanoma and will, therefore, greatly facilitate risk adapted therapy of malignant melanoma patients. In other words, based on a simple and cost-effective analysis of markers selected from this limited set, it is now possible to derive an assessment of the risk of an individual malignant melanoma patient for being a poor prognosis patient (disadvantageous course of disease), i.e. showing a high risk of disease recurrence or being a good prognosis patient (advantageous course of disease), i.e. showing a low risk of disease recurrence. Knowledge of these marker expression profiles additionally allows risk-adapted treatment, which is of benefit for patients with malignant melanoma. For example, a diagnosis of expression of MTAP enables practitioners to identify patients that may benefit from interferon treatment, therefore providing a new basis for a clear targeted use of this expensive immunotherapeutic agent and prevention of a considerable rate of serious side effects.

In particular, using tissue microarrays (TMA), samples of 364 patients with primary malignant melanoma were retrospectively analyzed. A panel of 70 immunohistochemical (IHC) antibodies for proteins involved in cell cycle, apoptosis, DNA mismatch repair, differentiation, proliferation, cell adhesion, signaling and metabolism was investigated. A marker selection procedure based on univariate Cox regression and multiple testing correction was employed to correlate the IHC expression data with the clinical follow-up (overall and recurrence-free survival). The model was thoroughly evaluated with two different cross validation experiments, a permutation test and a multivariate Cox regression analysis. The predictive power of the identified marker signature was validated on a second independent external test cohort (n=225), thus rendering the total of patients 589. This study adheres to the reporting recommendations for tumour marker prognostic studies, i.e. it implements the REMARK guidelines (J Natl Cancer Institute 2005; 97:1180-4).

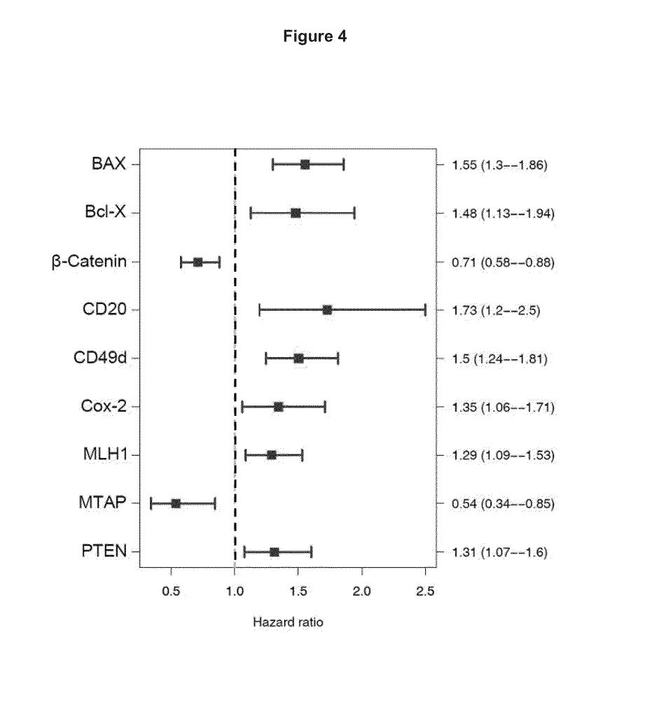

The prognostic power of these 70 markers was assessed, yielding the 11 markers MTAP, PTEN, Bax, Bcl-X, β-Catenin, CD20, Cox-2, CD49d, MLH1, TOP2A and Frizzled 7, which were significantly associated with overall survival. While all eleven markers are significantly associated with overall survival, it was possible to further reduce the number of markers required for a prognostic assessment to nine (MTAP, PTEN, Bax, Bcl-X, β-Catenin, CD20, Cox-2, CD49d and MLH1) based on the Cox regression coefficients and multiple testing correction with FDR. These nine markers were correlated with death from any cause. Two of these markers were protective markers (associated with a hazard ratio of less than 1.00) and seven were risk markers (associated with a hazard ratio of more than 1.00) (FIG. 4).

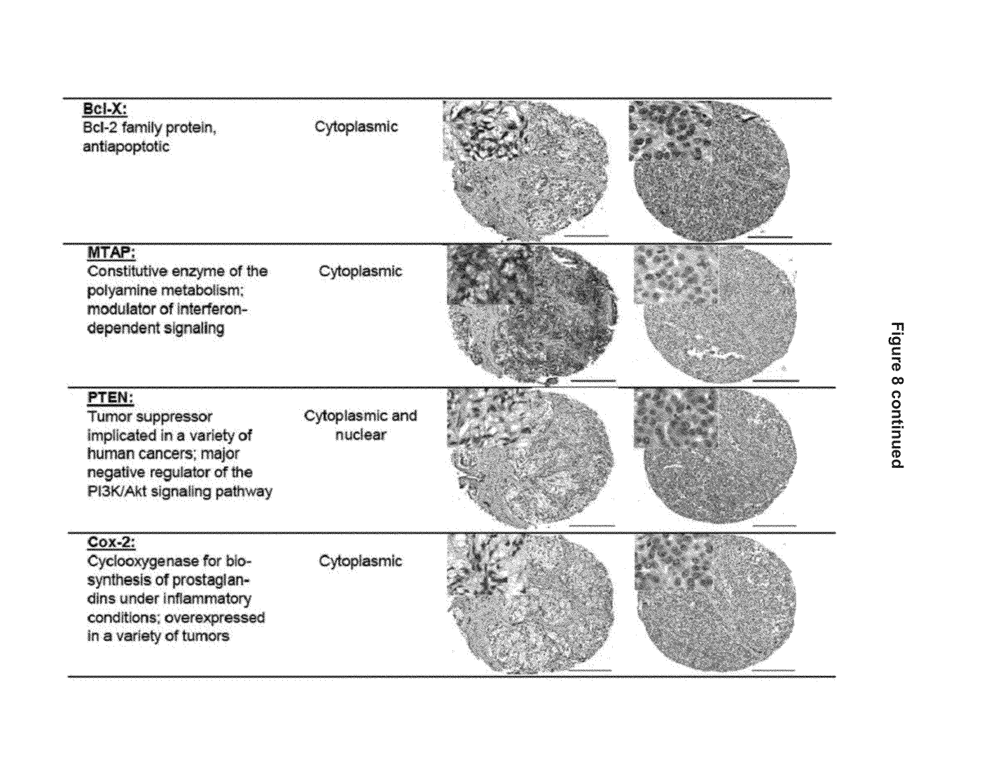

For the sake of clinical feasibility and cost saving, any marker set suitable for routine clinical assessment should comprise a limited number of markers. It was therefore the aim to provide a maximum of prognostic and therapeutically relevant information by as few markers as possible combined in a clear signature. Accordingly, it was shown that a further reduction of the nine-marker signature still leads to prognostic and therapeutically relevant information. As a proof of principle, this was carried out using a seven-marker signature (Bax, Bcl-X, β-Catenin, CD20, COX-2, MTAP, PTEN), which was found to also provide prognostic and therapeutically relevant information. Immunohistochemically stained TMA specimens illustrating the seven-marker signature for one patient with high-risk and one patient with low-risk melanoma is shown in FIG. 8.

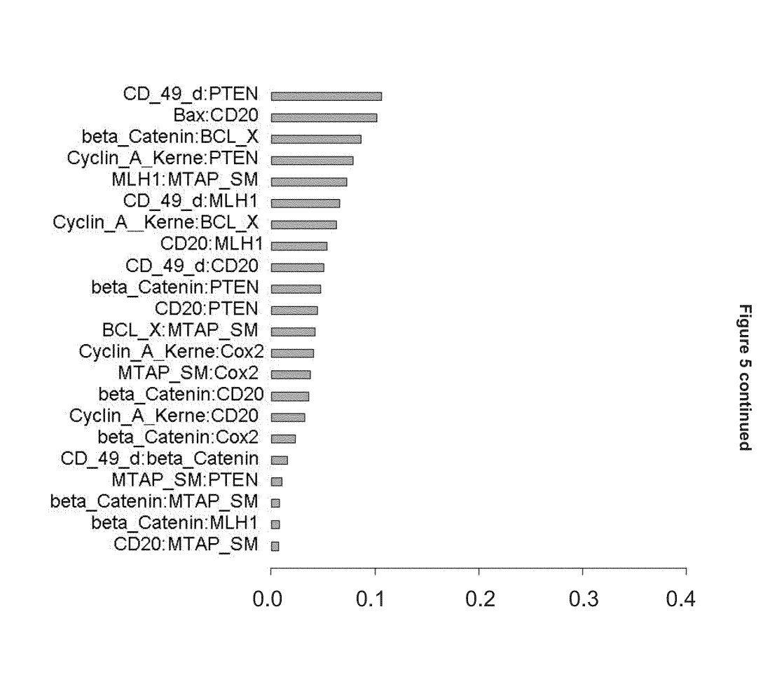

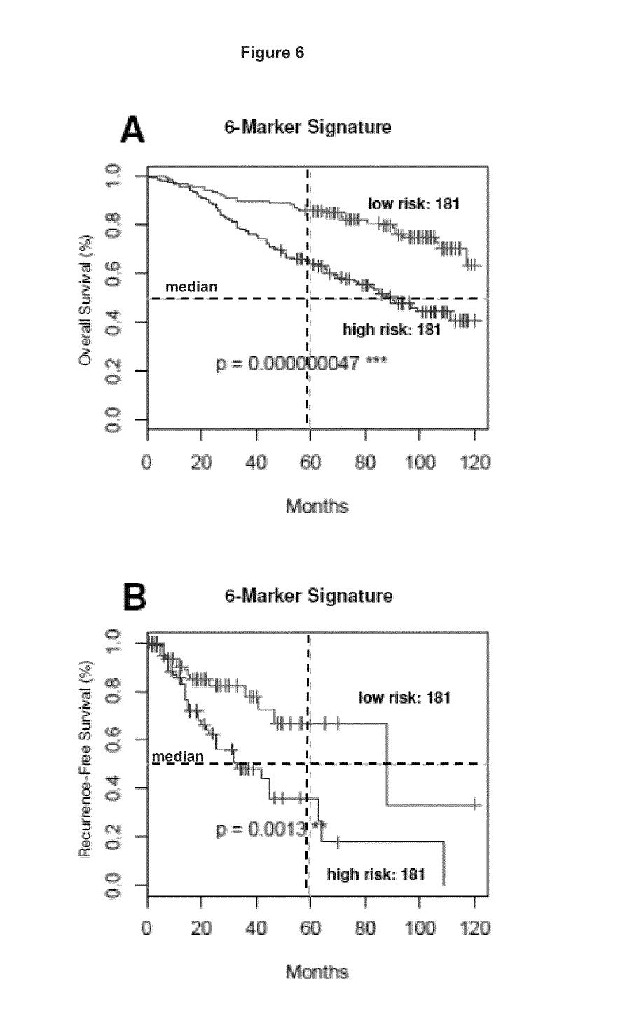

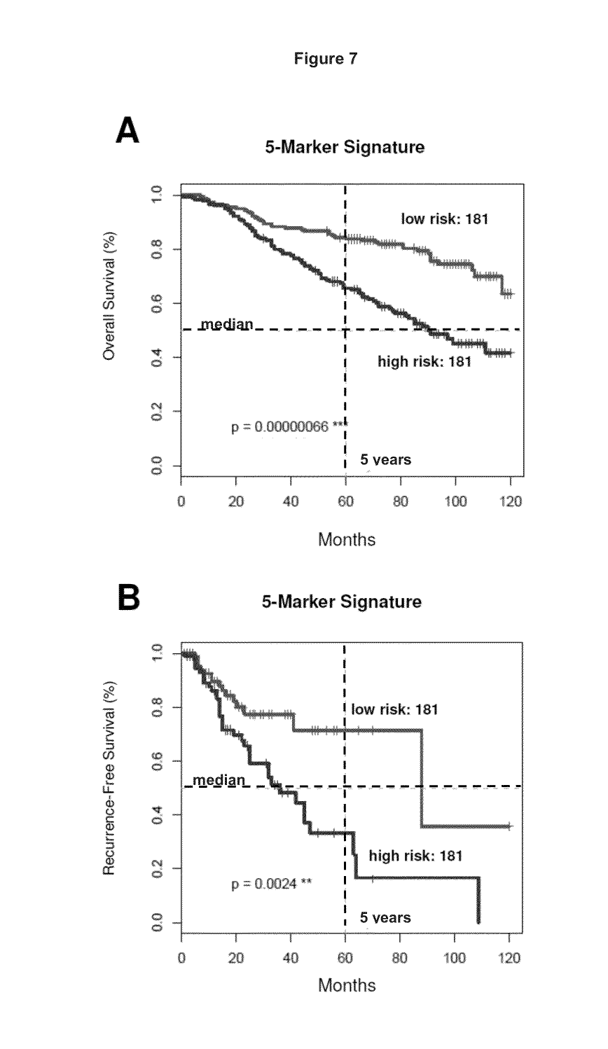

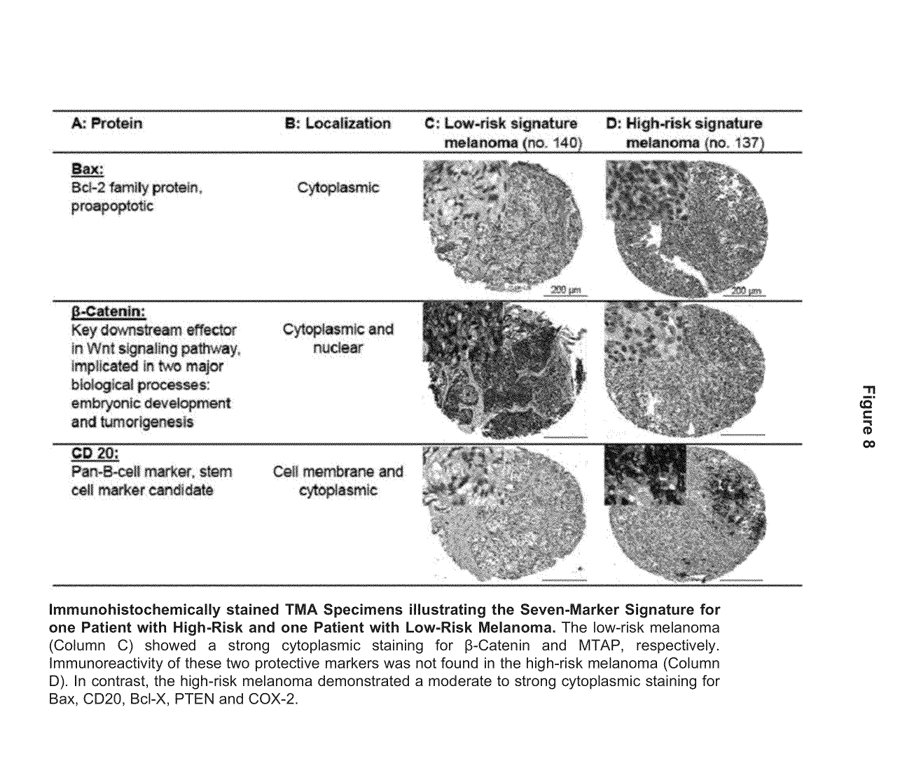

Moreover, further reduction of the seven-marker set by one and two marker(s), respectively, i.e. CD20 and PTEN, resulted in a six-/five-marker set that significantly correlated with overall and recurrence-free survival, as shown in Examples 5 and 6, and FIGS. 6 and 7 below.

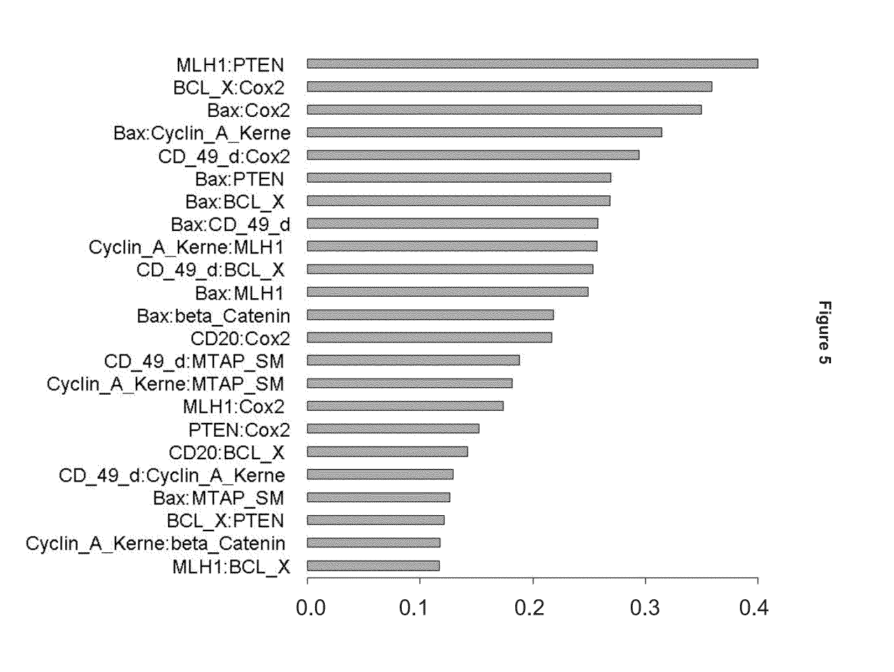

The data obtained for the seven-marker signature can reasonably be extended to marker sets comprising only five marker or six markers, due to the high coincidence and correlation of some of the markers, such as e.g. Bax and Cox-2 expression in the melanoma samples analyzed (see FIG. 5). In other words, when a highly correlated first marker is comprised in the set of markers analyzed, then the information provided by a second marker correlated to said first marker might be limited and, consequently, such markers can be omitted.

With a total of 24,674 punch specimens of primary malignant melanoma analyzed by IHC, this TMA study is more comprehensive than previous studies described in the art (to the inventors best knowledge). The detected signature might serve as a prognostic tool enabling physicians to selectively choose, at the time of diagnosis and initial surgery, the subset of high recurrence risk Stage I-II patients for adjuvant therapy. Selective treatment of those patients that are more likely to develop distant metastatic disease could potentially lower the burden of untreatable metastatic melanoma and promote the therapeutic management of malignant melanoma.

Means and methods to derive a risk estimate are well known in the art and, based on the information provided in accordance with the present invention, the skilled person is able to derive a risk estimation.

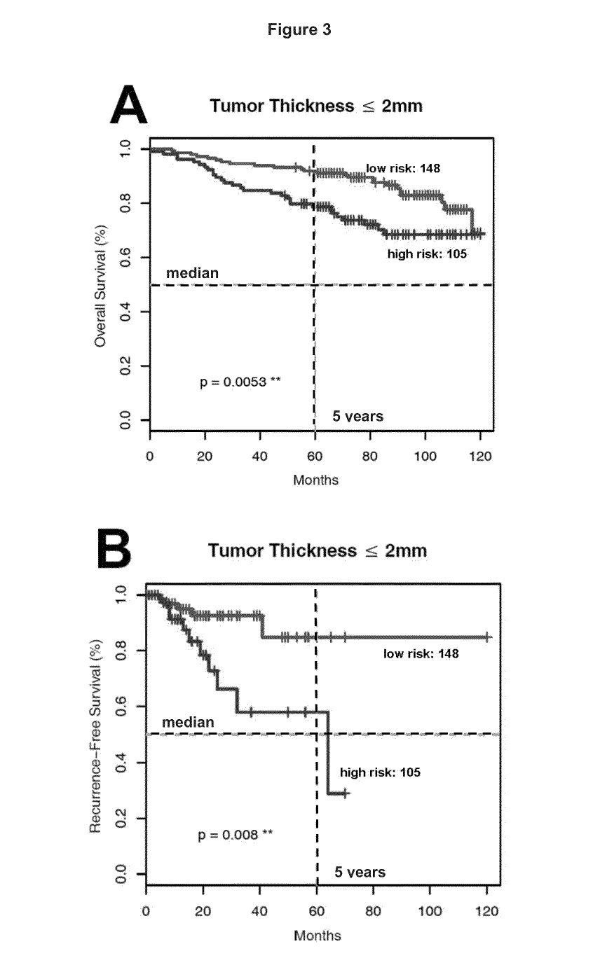

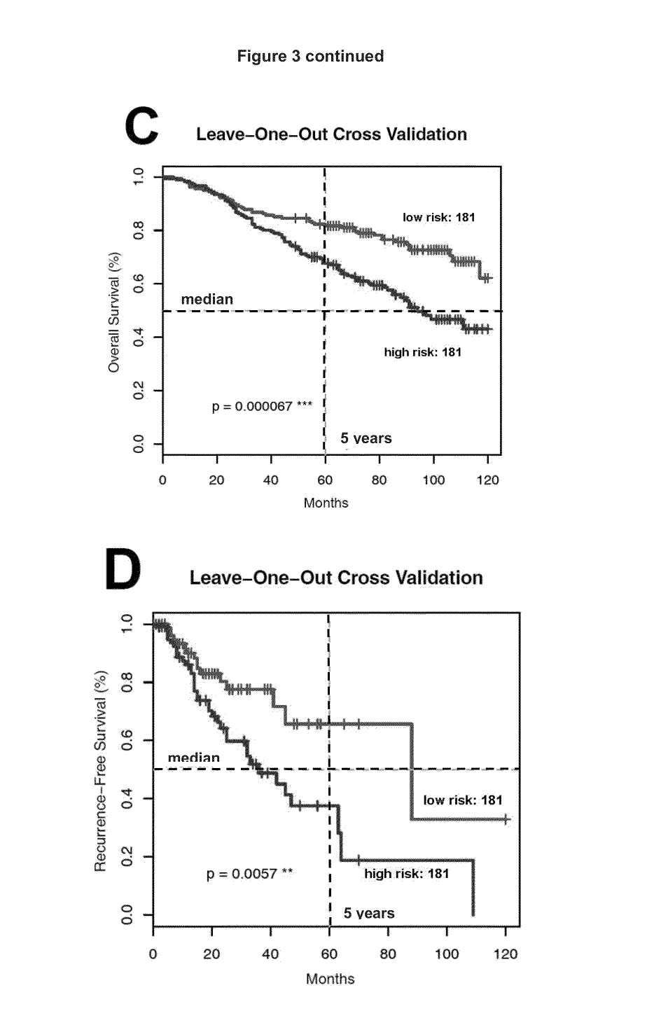

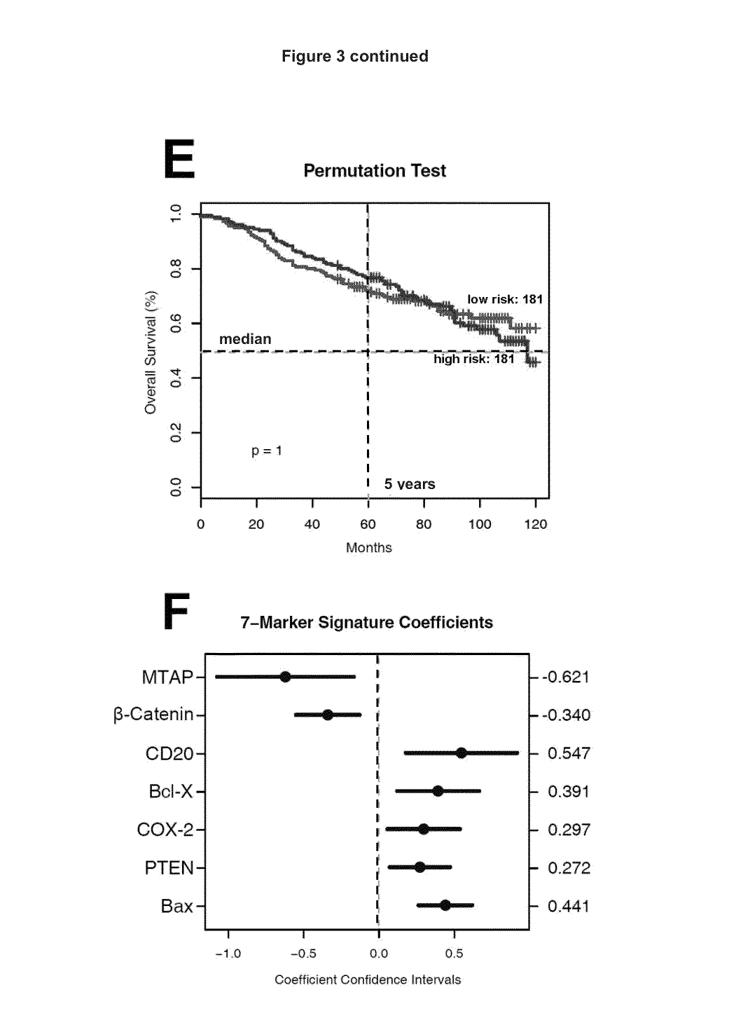

One exemplary method is based on the following prognostic score calculation:

$$score ( x ) = ( {\sum }_{i = 1}^{D} ( {β}_{i} {x}_{i} ) {α}_{i } ) \text{/} ( {\sum }_{i = 1}^{D} {α}_{i} ) , \\ {α}_{i} = \{ \begin{matrix}1 , & if {x}_{i} exists \\ 0 , & if {x}_{i} is missing\end{matrix}$$

wherein D is the number of markers analyzed, βᵢ are the coefficients of the univariate Cox model, e.g. −0.621 for MTAP, −0.34 for β-Catenin, 0.547 for CD20, 0.391 for Bcl-X, 0.297 for COX-2, 0.272 for PTEN, 0.407 for CD49d, 0.254 for MLH1 and 0.441 for Bax as shown in FIG. 3F and xᵢ is the value determined for the individual marker i in patient x. αᵢ corrects for markers not present in said patient or not evaluated. Based on this score calculation, a patient is diagnosed to have an advantageous course of disease when the score is below a reference score and to have a disadvantageous course of disease when the score is above said reference score.

In accordance with the present invention, the term “reference score” relates to a cut-off point above or below which a diagnosis of the course of disease can be made, i.e. as advantageous or disadvantageous course. Said reference score may for example be a mean, i.e. average, score determined based on a malignant melanoma patient cohort comprising patients with advantageous course of disease as well as disadvantageous course of disease without any bias towards one of these groups. Such un-biased patient cohorts will be available to hospitals and can be analyzed to derive the reference score. Preferably, a prognostic score significantly below the reference score is indicative of an advantageous course of disease and a prognostic score significantly above the reference score is indicative of a disadvantageous course of disease.

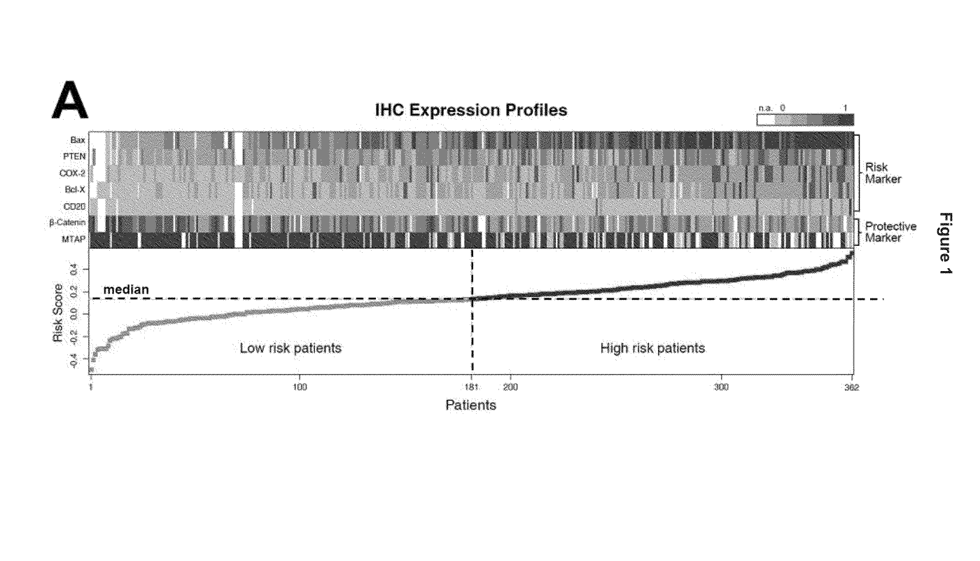

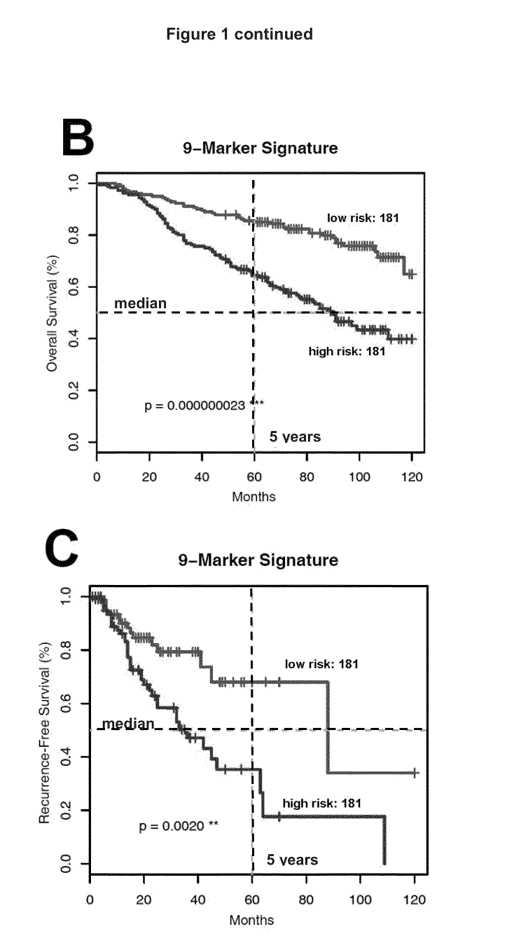

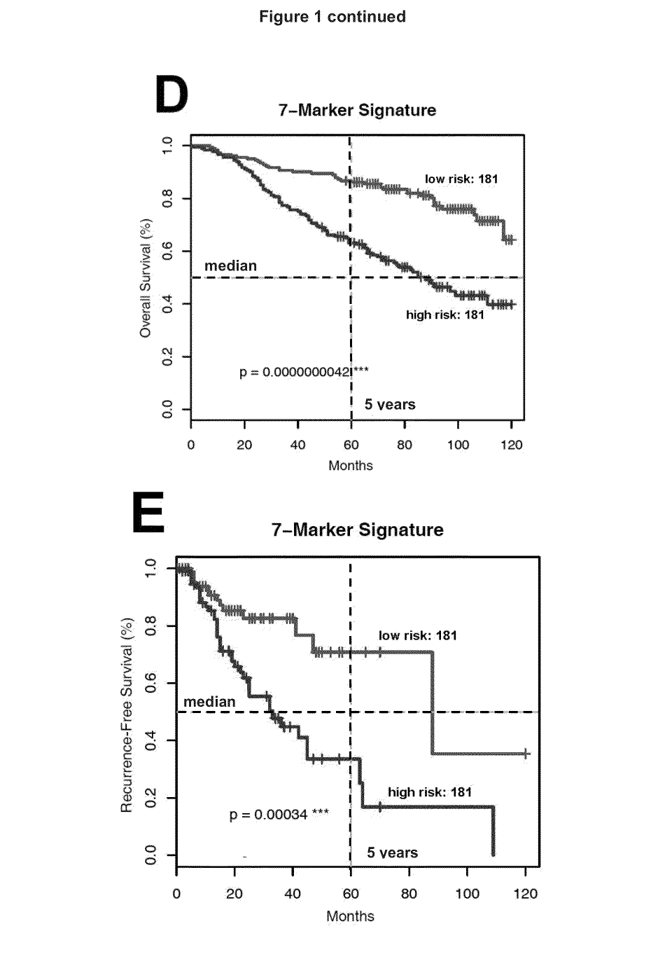

For example, based on the above score calculation and on a scale for x, ranging from −0.5 to +0.5, a patient is diagnosed to have an advantageous course of disease when the score based on the seven-marker signature (Bax, Bcl-X, β-Catenin, CD20, COX-2, MTAP, PTEN) is below 0.1346739 and to have a disadvantageous course of disease when the score is above 0.1346739, as shown in FIG. 1A.

Alternatively, the skilled person may initially determine in a sufficiently large patient group, such as for example at least 10, more preferably at least 75 and most preferably at least 100 patients, the presence and amount of the markers MTAP, PTEN, Bax, Bcl-X, β-Catenin, CD20, Cox-2, CD49d and MLH1. Preferably, the patient group is a representative group of malignant melanoma patients predicted by conventional methods to be poor prognosis or good prognosis patients. The data obtained from this group may then be correlated with disease progression, as detailed in the examples below. For example, after measurement of at least 10, more preferably at least 75 and most preferably at least 100 patients in a heterogeneous prognostic group and determination of the above mentioned marker values, correlation of the follow up data with the marker values using the univariate Cox model results in the derivation of specific coefficients. The calculated scores (sum of the marker values multiplied with the coefficients) can be sorted due to their magnitude. The cut offs should be selected due to clinical relevance. For example the magnitudes may be split at the 50th percentile, whereas a lower score is indicative for good prognosis and a higher score is indicative for poor prognosis. Alternatively, the patients may be grouped into more than two groups, depending on the clinical information required. It will be understood by the skilled person that such an initial determination of the presence and amount of markers does not need to be carried out every time but may instead be carried out when first establishing the method of predicting the course of malignant melanoma in patients. The data obtained by such initial experiments may also be stored in databases accessible to other researchers, thus obviating the need for these researchers to establish such initial data.

Alternatively, the risk of an individual patient may also be evaluated based on a simplified score calculation wherein the coefficients from the univariate Cox proportional hazard models are used in a weighted linear combination to predict the risk score for each patient as shown in FIG. 3F. Such a simplified score calculation would be: −0.6*MTAP−0.3*β-Catenin+0.5*CD20+0.4*BCLX+0.4*Bax+0.3*PTEN+0.3*COX2. In other words, the score obtained for MTAP is multiplied with −0.6, the score obtained for β-Catenin is multiplied with −0.3, the score obtained for CD20 is multiplied with 0.5 and so on. The sum of all these weighted scores provides a prognostic risk score for each patient. For example and as shown in the appended examples, when a scoring system from 0 to 4 was employed, a risk score below 0.135 was found to be indicative of a good prognosis for said patient while a risk score above 0.135 was found to be indicative of a poor prognosis for said patient.

In a preferred embodiment of the method of the invention, the at least five biomarkers include PTEN and/or MTAP.

Thus, the set of biomarkers employed in accordance with this preferred embodiment comprises at least PTEN or MTAP, more preferably it comprises PTEN and MTAP.

MTAP expression was shown in accordance with the present invention to be the strongest marker for a favourable disease outcome (coefficient of −0.621) and is, furthermore, of therapeutic relevance. In the adjuvant treatment of malignant melanoma, interferon alpha is currently the only clinically accepted therapeutic agent providing a significant (recurrence-free) survival benefit for a small but distinct percentage of patients (Ascierto, P. A. et al., 2008). On account of the serious side effects and the high costs of the therapy, it is advantageous to determine those patients with a realistic chance to benefit from interferon treatment. It has recently been shown that there is a clear association between MTAP expression in the primary melanoma and melanoma progression and, even more importantly, response to interferon treatment (Wild, P. J. et al., 2006; Zhang, S. et al., 2010; Meyer, S. et al., 2010). Biomarkers like MTAP might therefore enable practitioners to assess which patients may benefit from interferon treatment and could thus provide a new basis for a clear targeted use of this expensive immunotherapeutic agent and prevent the serious side effects associated with the treatment with interferon.

The tumor suppressor phosphatase and tensin homolog PTEN was identified as another signature protein. PTEN counteracts one of the most critical cancer promoting pathways (Zhang, S. et al., 2010,) the phosphatidylinositol 3-kinase (PI3K)/Akt signaling pathway. An established consequence of PTEN inactivation is the constitutive aberrant activation of the PI3K-signaling pathway that drives uncontrolled cell growth, proliferation, and survival (Zhang and Yu, 2010). Thus, including PTEN in the analysis not only provides a diagnostic tool, but may also be of predictive relevance. In general, cancer cells contain multiple genetic and epigenetic abnormalities. Despite this complexity, their growth and survival can often be impaired by the inactivation of a single oncogene. This phenomenon, called “oncogene addiction,” provides a rationale for molecular targeted therapy. The efficacy of this strategy requires novel methods, including integrative genomics and systems biology, to identify the state of oncogene addiction (i.e., the “Achilles heel”) in specific cancers. Combination therapy may also be required to prevent the escape of cancers from a given state of oncogene addiction (Weinstein and Joe 2008). Thus, including PTEN in the analysis not only provides a diagnostic tool, but also is also of therapeutic relevance.

In another preferred embodiment of the method of the invention, at least seven biomarkers are determined.

In a more preferred embodiment, the at least 7 biomarkers are MTAP, PTEN, Bax, Bcl-X, β-Catenin, CD20 and Cox-2.

In accordance with the present invention, it has been shown that this set of seven biomarkers is closely associated with the prognosis of patients with malignant melanoma and may therefore serve as an independent predictor for overall and recurrence-free survival in patients with malignant melanoma.

Among the 362 patients of the primary cohort, patients with said high-risk seven-marker signature had a shorter median overall survival than the patients with a low-risk seven-marker signature (88 months versus not reached) and the difference between the two patient groups was highly significant (p=0.0000000042) (FIG. 1D). The high-risk seven-marker signature was associated with a median recurrence-free survival of 33 months, whereas the low-risk seven-marker signature was associated with a median recurrence-free survival of 88 months (LRT p=0.00034) (FIG. 1E). According to multivariate Cox regression analysis, the seven-marker risk score, tumour thickness, sex, and age were significantly associated with death from any cause among the 356 patients (6 observations were deleted due to missing values) (Table 1). Furthermore, a subgroup analysis of 253 patients with a tumour depth of 2 mm revealed that those 148 patients with a high-risk marker signature had a significant (p=0.0053) shorter overall survival (FIG. 3A) and recurrence-free survival (p=0.008) than the 105 patients with a low-risk marker signature (FIG. 3B).

TABLE 1
Clinical Characteristics of the Primary Cohort of Patients with MM (TMA 1)

| | High risk | Low risk | p-Value: | Multivariate Cox Regression Analysis | |
|---|---|---|---|---|---|
| | (N = 181) | (N = 181) | high vs. low risk | Hazard ratio(95% CI) | p-Value |
| 7-Marker risk score | 0.267 ± 0.092 | 0.0017 ± 0.12 | <<0.0001 *¹ | 13.54 (4.27-42.97) | 0.0000098 *** |
| Age - yr | 59.5 ± 15.0 | 57.7 ± 14.9 | 0.263 *¹ | 1.03 (1.02-1.05) | 0.0000027 *** |
| Sex - no. of patients (%) | | | | | |
| Male | 105 (58) | 89 (49.2) | 1 *² | 1.98 (1.36-2.89) | 0.00034 *** |
| Female | 76 (42) | 92 (50.8) | | | |
| Tumor thickness - mm | 2.52 ± 2.38 | 1.40 ± 2.21 | 0.00000646 *¹ | 1.17 (1.12-1.23) | 0.00000000023 *** |

*¹ Welch two sample t-test
*² Fisher's exact test
*** p-Value < 0.001

Comparing high-risk patients (first column) with low-risk patients (second column) based on their seven-marker risk score shows significant difference in tumour thickness (p<0.001) and no difference in sex (p=1) and age (p=0.263). Furthermore, hazard ratios and p-values are reported for a multivariate Cox regression model comprising all listed variables. Regarding overall survival the seven-marker risk score is statistically significant (p<0.001) independent of sex, age and tumour thickness. Continuous variables are reported with mean and standard deviation and categorical variables are listed with number of counts and percentages.

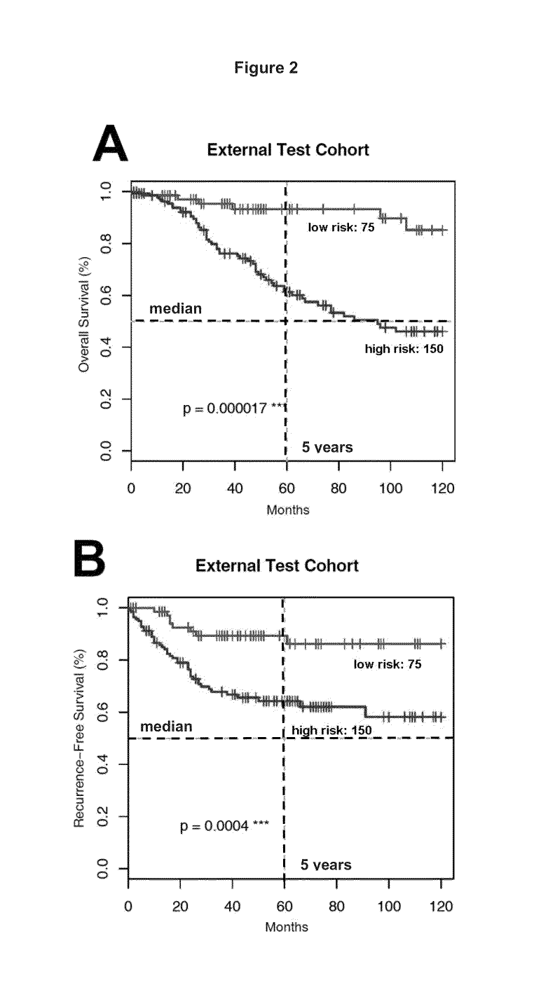

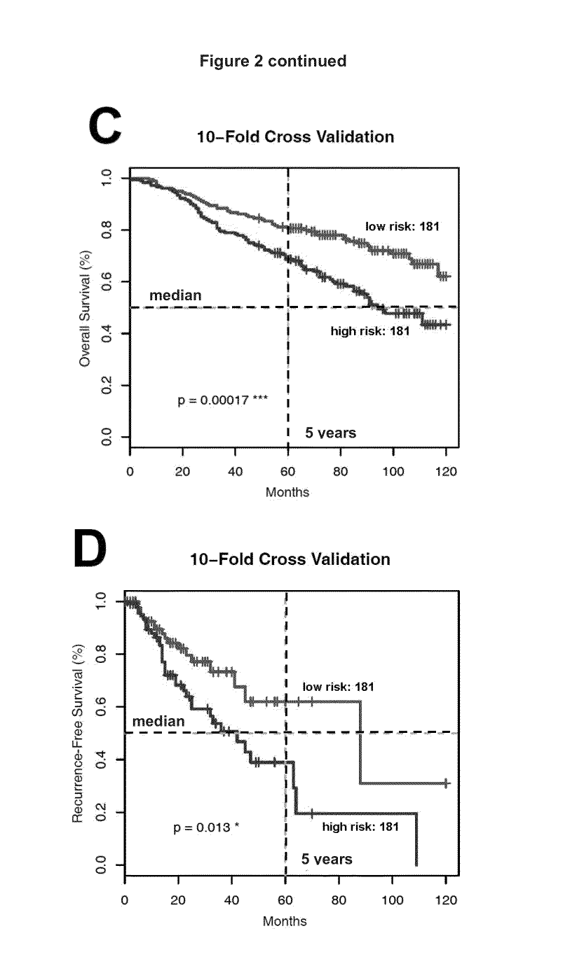

Notably, the predictive power of the signature was carefully validated and confirmed on a secondary, independent external test cohort including melanoma samples of 225 patients from a different hospital. In this external test cohort it was confirmed that patients with a high-risk marker signature had a significantly (p=0.000017) different survival expectance and shorter median overall survival compared to patients with a low-risk signature (95 months versus not reached) (FIG. 2A). According to multivariate Cox regression including sex, age, tumour thickness, ulceration and nodal status, the seven-marker signature was significantly associated with overall survival (p=0.0000098, Table 1). Additionally, the recurrence-free survival differed significantly between the two risk groups (p=0.004; FIG. 2B).

Thus, the seven-marker signature (Bax, Bcl-X, β-Catenin, CD20, COX-2, MTAP, PTEN) is closely associated with the prognosis of patients with malignant melanoma, as the signature was found to be an independent predictor for overall and recurrence-free survival in patients with malignant melanoma. The seven-marker signature could also predict high recurrence risk patients with localized primary malignant melanoma stage pT1-2 (tumour thickness≦2.00 mm) and worse prognosis. In particular, three of these markers (CD20, COX-2, MTAP) were shown to offer direct therapeutic implications.

In a further preferred embodiment of the method of the invention, at least nine biomarkers are determined.

By increasing the number of biomarkers analyzed, the sensitivity and specificity of the analysis can be increased.

In accordance with this embodiment, all nine biomarkers MTAP, PTEN, Bax, Bcl-X, β-Catenin, CD20, Cox-2, CD49d and MLH1 are determined.

It was found in accordance with the present invention that this set of nine biomarkers is closely associated with the prognosis of patients with malignant melanoma and may therefore serve as an independent predictor for overall and recurrence-free survival in patients with malignant melanoma.

Table 1 summarizes the characteristics of 362 patients in the study. Among these 362 patients of the primary cohort, tumours associated with high risk scores also expressed risk markers, whereas tumours associated with low risk scores expressed protective markers (FIG. 1A). Patients with a high-risk nine-marker signature had a lower median overall survival than patients with a low-risk nine-marker signature (90 months versus not reached) (FIG. 1B). Patients with tumours with a high-risk marker signature were associated with a lower median recurrence-free survival than patients with tumours with a low-risk gene signature (36 months versus 88) (FIG. 1C).

The present invention further relates to a method of preparing a tailored pharmaceutical composition for a patient having a malignant melanoma, the method comprising (i) determining in melanoma cells comprised in a sample obtained from said malignant melanoma the presence or amount of at least five biomarkers selected from the group comprising or consisting of MTAP, PTEN, Bax, Bcl-X, β-Catenin, CD20, Cox-2, CD49d and MLH1, wherein the absence or decreased amount of β-Catenin and MTAP and/or the presence or increased amount of PTEN, Bax, Bcl-X, CD20, Cox-2, CD49d and MLH1 is associated with a disadvantageous course of disease; (ii) deriving a treatment regimen for the individual patient based on the presence or amount of markers determined in step (i); and (iii) providing at least one pharmaceutical compound based on the treatment regimen derived in step (ii).

In accordance with this embodiment, the course of disease is determined for a patient having a malignant melanoma and, additionally, the data obtained by determining the presence or amount of at least five biomarkers selected from the group comprising or consisting of MTAP, PTEN, Bax, Bcl-X, β-Catenin, CD20, Cox-2, CD49d and MLH1 is employed to derive a treatment regimen for the individual patient, i.e. to prepare a tailored pharmaceutical composition.

The term “pharmaceutical composition”, as used herein, relates to a composition for administration to a patient, preferably a human patient. The pharmaceutical composition of the invention comprises a therapeutic compound, such as for example a compound selected from the compounds recited below, alone or in combination. It may, optionally, comprise further molecules capable of altering the characteristics of these compounds thereby, for example, stabilizing, modulating and/or activating their function. The composition may e.g. be in solid or liquid form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. By “pharmaceutically acceptable carrier” is meant a non-toxic solid, semisolid or liquid filler, diluent, encapsulating material or formulation auxiliary of any type. Examples of suitable pharmaceutically acceptable carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods.

These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. The skilled person knows that the effective amount of a pharmaceutical composition administered to an individual will, inter alia, depend on the nature of the compound. For example, if said compound is a polypeptide, the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day. Furthermore, if for example said compound is an iRNA agent, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of iRNA agent (e.g., about 4.4×1,016 copies) per kg of body weight, such as for example less than 1,500, 750, 300, 150, 75, 15, 7.5, 1.5, 0.75, 0.15, 0.075, 0.015, 0.0075, 0.0015, 0.00075 or 0.00015 nmol of iRNA agent per kg of body weight. The length of treatment needed to observe changes and the interval following treatment for responses to occur vary depending on the desired effect. The particular amounts may be determined by conventional tests which are well known to the person skilled in the art.

Pharmaceutical compositions of the invention may for example be administered orally, rectally, parenterally, intracisternally, intraperitoneally, topically (as by powders, ointments, drops or transdermal patch), bucally, or as a nasal spray. The term “parenteral” as used herein refers to modes of administration, which include intravenous, intramuscular, intrasternal, subcutaneous and intraarticular injection and infusion.

The therapeutic compound, in accordance with the present invention, may for example be selected from the group consisting of antibodies, aptamers, siRNAs, shRNAs, miRNAs, ribozymes, antisense nucleic acid molecules, and a small molecule. Therapeutic compounds further include but are not limited to, for example, peptides such as soluble peptides, including Ig-tailed fusion peptides and members of random peptide libraries (see, e.g., Lam et al. (1991) Nature 354: 82-84; Houghten et al. (1991) Nature 354: 84-86) and combinatorial chemistry-derived molecular libraries made of D- and/or L-configuration amino acids or phosphopeptides (e.g., members of random and partially degenerate, directed phosphopeptide libraries, see, e.g., Songyang et al. (1993) Cell 72: 767-778).

The term “antibody” as used in accordance with the present invention comprises polyclonal and monoclonal antibodies, as well as derivatives or fragments thereof, which still retain the binding specificity. Antibody fragments or derivatives comprise, inter alia, Fab or Fab′ fragments as well as Fd, F(ab′)₂, Fv or scFv fragments; see, for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also includes embodiments such as chimeric (human constant domain, non-human variable domain), single chain and humanised (human antibody with the exception of non-human CDRs) antibodies. The term antibodies also encompasses peptidomimetics.

Various techniques for the production of antibodies are well known in the art and described, e.g. in Harlow and Lane (1988) and (1999), loc. cit. Further, techniques described for the production of single chain antibodies (see, inter alia, U.S. Pat. No. 4,946,778) can be adapted to produce single chain antibodies specific for the target of this invention. Also, transgenic animals or plants (see, e.g., U.S. Pat. No. 6,080,560) may be used to express (humanized) antibodies specific for the target of this invention. Most preferably, the antibody is a monoclonal antibody, such as a human or humanized antibody. For the preparation of monoclonal antibodies, any technique which provides antibodies produced by continuous cell line cultures can be used. Examples for such techniques are described, e.g. in Harlow and Lane (1988) and (1999), loc. cit. and include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to an epitope of the biomarkers (Schier, Human Antibodies Hybridomas 7 (1996), 97-105; Malmborg, J. Immunol. Methods 183 (1995), 7-13). It is also envisaged in the context of this invention that the term “antibody” comprises antibody constructs which may be expressed in cells, e.g. antibody constructs which may be transfected and/or transduced via, inter alia, viruses or plasmid vectors.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

More specifically, aptamers can be classified as nucleic acid aptamers, such as DNA or RNA aptamers, or peptide aptamers. Whereas the former normally consist of (usually short) strands of oligonucleotides, the latter preferably consist of a short variable peptide domain, attached at both ends to a protein scaffold.

Nucleic acid aptamers are nucleic acid species that, as a rule, have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers usually are peptides or proteins that are designed to interfere with other protein interactions inside cells. They typically consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys- loop in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

The term “peptide” as used herein describes a group of molecules consisting of up to 30 amino acids, whereas the term “protein” as used herein describes a group of molecules consisting of more than 30 amino acids. Peptides and proteins may further form dimers, trimers and higher oligomers, i.e. consisting of more than one molecule which may be identical or non-identical. The corresponding higher order structures are, consequently, termed homo- or heterodimers, homo- or heterotrimers etc. The terms “peptide” and “protein” (wherein “protein” is interchangeably used with “polypeptide”) also refer to naturally modified peptides/proteins wherein the modification is effected e.g. by glycosylation, acetylation, phosphorylation and the like. Such modifications are well-known in the art.

Antibodies or aptamers may further be used as a targeting moiety to deliver therapeutically active compounds, such as known anti-cancer drugs (e.g. chemotherapeutic agents such as Dacarbazine, Fotemustine or Cisplatin), to the malignant melanoma cells of a patient.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene of which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1-5 nucleotides, more preferably from 1-3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have 2-nt 3′ overhangs on either end. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to typically silence gene expression via RNA interference. shRNA can for example use a vector introduced into cells, in which case the U6 promoter is utilized to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. Preferably, si/shRNAs to be used in the present invention are chemically synthesized using conventional methods that, for example, appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules which, as endogenous RNA molecules, regulate gene expression. Binding to a complementary mRNA transcript triggers the degradation of said mRNA transcript through a process similar to RNA interference. Accordingly, miRNA may be employed as an inhibitor of the of the biomarkers in accordance with the present invention.

A ribozyme (from ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in the last 10 years. The hammerhead ribozymes are characterized best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each usually with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

A recent development, also useful in accordance with the present invention, is the combination of an aptamer recognizing a small compound with a hammerhead ribozyme. The conformational change induced in the aptamer upon binding the target molecule is supposed to regulate the catalytic function of the ribozyme.

The term “antisense nucleic acid molecule” is known in the art and refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

A “small molecule” according to the present invention may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity, can be identified and verified in in vivo assays such as in vivo high-throughput screening (HTS) assays. Such small molecules may be particularly suitable to inhibit protein-protein-interaction by blocking specific bindings sites of the target molecule. Suitable small molecules currently employed in the treatment of cancer include, without being limiting, small molecule inhibitors for inhibiting the Bcl-2 apoptosis inhibitor family (Azmi, and Mohammad, 2009).

Also encompassed herein are modified versions of these therapeutic compounds.

The term “modified versions of these therapeutic compounds” in accordance with the present invention refers to versions of the compounds that are modified to achieve i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, metabolism and excretion), and/or vii) modified physico-chemical parameters (solubility, hygroscopicity, color, taste, odor, stability, state), and/or viii) improved general specificity, organ/tissue specificity, and/or ix) optimised application form and route by (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (k) conversion of alkyl substituents to cyclic analogues, or (L) derivatisation of hydroxyl groups to ketales, acetales, or (m) N-acetylation to amides, phenylcarbamates, or (n) synthesis of Mannich bases, imines, or (o) transformation of ketones or aldehydes to Schiff's bases, oximes, acetales, ketales, enolesters, oxazolidines, thiazolidines; or combinations thereof.

The various steps recited above are generally known in the art. They include or rely on quantitative structure-action relationship (QSAR) analyzes (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

The term “tailored pharmaceutical composition” in accordance with the present invention, relates to a pharmaceutical composition that is adjusted to the individual needs of a particular patient. In other words, a tailored pharmaceutical composition is a patient-specific medication. For the practitioner, the assessment of the markers in accordance with the invention provides a helpful tool to answer the crucial question “whom to treat, and how to treat”, especially in the adjuvant setting after surgical excision of early-stage and localized primary malignant melanoma (Stage I to IIa).

Several of the markers employed in accordance with the present invention have previously been shown to be suitable targets or indicators in the treatment of cancers. Thus, therapeutic compounds targeting said markers or targeting pathways associated with said markers are already available in the art. For example, CD20, COX-2 and MTAP are three markers of the present invention that offer direct therapeutic implications, since the corresponding drugs have been approved by the FDA.

The CD20-antigen is known to be an effective therapeutic target in the treatment of patients with CD20-positive B-Cell-Non-Hodgkin-Lymphomas. For example, the monoclonal chimeric antibody Rituximab has been described for immunotherapy (Avivi, I. et al., 2003). The antibody binds specifically with CD20-antigen presented on the surface of normal and malignant B-lymphocytes and causes a cell- and complement-mediated cytotoxic death of these cells. According to a small phase II pilot trial in stage IV melanoma patients recently presented at the ASCO meeting (2010), the anti-CD20-antibody Rituximab may potentially be suitable for immunotargeting of CD20-positive melanoma subpopulations. Consequently, the presence of CD20 in melanoma cells of a patient as determined with the method of the present invention is indicative for a therapeutic treatment of said patient with compounds targeting CD20, such as for example Rituximab. Further CD20 inhibitors are for example, the yttrium-[90]-labeled 2138 murine antibody designated Y2B8 (U.S. Pat. No. 5,736,137); murine IgG2a 131 optionally labeled with 131 I to generate the 131 I-B1 antibody (BEXXAR®) (U.S. Pat. No. 5,595,721); murine monoclonal antibody 1F5 (Press et al. Blood 69(2): 584-591 (1987)); chimeric 2H7 antibody (U.S. Pat. No. 5,677,180); and monoclonal antibodies L27, G28-2, 93-1 133, B—Cl or NU-B2 available from the International Leukocyte Typing Workshop (Valentine et al., In: Leukocyte Typing III (McMichael, Ed., p. 440, Oxford University Press (1987)).

Cyclooxygenase 2 represents another promising therapeutic target. Cyclooxygenases (COXs) catalyze the first rate-limiting step in the conversion of arachidonic acid to prostaglandins. In contrast to COX-1, the COX-2 isoenzyme is not detectable in most normal tissues and rapidly induced by various stimuli such as inflammatory reactions (Hla, T. et al., 1992). It is also expressed in various tumour types and levels of COX-2 expression have been shown to correlate with invasiveness and prognosis in some tumour entities, including epithelial and melanocytic skin cancer (Meyer, S. et al., 2009; Denkert, C. et al., 2001). Epidemiological studies showed that prolonged COX-2 inhibition through acetylsalicylic acid or other nonsteroidal anti-inflammatory drugs (NSAIDs) might offer some protection against colon cancer and some other malignancies (Thun, M. J. et al., 2004). So far the benefit of COX-2-inhibitors has not been studied in the adjuvant treatment of early-stage melanomas to prevent metastasis. In the second-line treatment of advanced metastatic melanoma disease, however, a survival benefit was shown for targeted combined therapy using COX-2-inhibitors and PPARG-agonists for anti-inflammatory treatment together with low-dose metronomic chemotherapy (Reichle, A. et al., 2007). Considering this observation and the fact that melanoma patients with COX-2-positive primary tumours bear a significantly higher risk of tumour recurrence (Meyer, S. et al., 2009,) it is concluded that the presence of COX-2 in melanoma cells of a patient as determined with the method of the present invention is indicative for a therapeutic treatment of said patient with compounds targeting COX-2, such as for example COX-2 inhibitors including, but not limited, Celecoxib, Etoricoxib or Parecoxib for primary adjuvant treatment of these patients.

Furthermore, in the adjuvant treatment of malignant melanoma, interferon alpha is currently the only clinically accepted therapeutic agent providing a significant (recurrence-free) survival benefit for a small but distinct percentage of patients (Ascierto, P. A. et al., 2008). On account of the serious side effects and the high costs of the therapy, it is advantageous to determine those patients with a realistic chance to benefit from interferon treatment. It has recently been shown that there is a clear association between MTAP expression in the primary melanoma and melanoma progression and, even more importantly, response to interferon treatment (Wild, P. J. et al., 2006; Zhang, S. et al., 2010; Meyer, S. et al., 2010). Biomarkers like MTAP might therefore enable practitioners to assess which patients may benefit from interferon treatment and could thus provide a new basis for a clear targeted use of this expensive immunotherapeutic agent and prevent the serious side effects associated with the treatment with interferon.

Also Bcl-X has been targeted in preclinical tests and several targeting agents are in the clinical testing phase by now (Azmi, A. S. et al., 2009.) Bcl-X is related to the anti-apoptotic Bcl-2 protein family. Over-expression of these anti-apoptotic proteins protects cancer cells against death signals of apoptosis. Interestingly, tumours expressing high levels of Bcl-2 or Bcl-X are often found to be resistant to chemotherapeutic agents or radiation therapy (Heere-Ress, E. et al., 2002). In recent years, there has been an exponential growth in the identification and synthesis of non-peptidic cell permeable “small molecule inhibitors” (SMIs) against anti-apoptotic proteins like Bcl-2 or Bcl-X (see e.g. Azmi and Mohammad 2009). SMIs inhibit distinct protein-protein interactions by blocking specific binding sites of the target molecule, thus supporting the apoptotic machinery (Azmi, A. S. et al., 2009). Inhibition of Bcl-X may exert a synergistic effect with conventional treatments like chemo- or radiation therapy and with respect to melanoma therapy, this effect would be a decisive therapeutic success.

For PTEN oncogenic pathway addiction, as described herein above, has been described in detail in the literature, for example in Weinstein and Joe 2008, Zhang and Yu 2010, Mirmohammadsadegh et al. 2006, Lahtz et al. 2010 or Zhou et al. 2000.

In accordance with the present invention, the marker signature represents a highly promising clinical tool to predict a patient's prognosis. Most importantly, the marker signature is expected to improve the clinical management and adjuvant treatment of early-stage malignant melanoma with a high risk of recurrence. In the treatment of advanced metastatic melanoma, novel immune-based anti-tumour therapies targeting signal transduction pathways or tumour immunity barriers by monoclonal antibodies like selective BRAF inhibitors (Hauschild, A. et al., 2009) or anti-cytotoxic T-lymphocyte antigen 4 (CTLA-4) antibodies (Hodi, F. S. et al., 2010) have already entered clinical studies. This promising therapeutic option in the treatment of advanced metastatic malignant melanoma development, together with the set of molecular markers identified in accordance with the present invention is expected to provide new risk-oriented indications for an individualized targeted anti-tumour therapy of malignant melanoma.

In a preferred embodiment of the method of preparing a tailored pharmaceutical composition, the at least five biomarkers include CD20, Cox-2 and/or MTAP.

Thus, the set of biomarkers employed in accordance with this preferred embodiment comprises at least CD20, or Cox-2, or MTAP, or CD20 and Cox-2, or CD20 and MTAP, or Cox-2 and MTAP, or CD20 and Cox-2 and MTAP.

In another preferred embodiment of the method of preparing a tailored pharmaceutical composition, at least seven biomarkers are determined.

In a more preferred embodiment of the method of preparing a tailored pharmaceutical composition, the at least seven biomarkers are MTAP, PTEN, Bax, Bcl-X, 0-Catenin, CD20 and Cox-2.

In a further preferred embodiment of the method of preparing a tailored pharmaceutical composition, at least nine biomarkers are determined.

In accordance with this embodiment of the method of preparing a tailored pharmaceutical composition, all nine biomarkers MTAP, PTEN, Bax, Bcl-X, 0-Catenin, CD20, Cox-2, CD49d and MLH1 are determined.

In a further more preferred embodiment of the method(s) of the invention, the sample is obtained from the primary tumour, a lymph node or a metastasis.

The term “primary tumour” in accordance with the present invention refers to a malignant tumour (also referred to herein as a cancer) at a first site, i.e. in a first organ or part of the body. In general, when the area of cancer cells at the originating site become clinically detectable, it is referred to as a primary tumour. In the present case of malignant melanoma, said primary tumour is a malignant tumour of melanocytes, which are present in the skin (i.e. the primary tumour is skin cancer) but also in the mucous membrane and the eye. Some cancer cells also acquire the ability to penetrate and infiltrate surrounding normal tissues in the local area, forming a new tumour. The newly formed “daughter” tumour in the adjacent site within the tissue is called a local metastasis while the formation of a new tumour in a non-adjacent site is called a distant metastasis.

In accordance with the present invention, the term “lymph node” refers a small organ of the immune system that is important in the proper functioning of the immune system, as it acts as a filter or trap for foreign particles. Lymph nodes are widely distributed throughout the body including the armpit and stomach/gut and linked by lymphatic vessels. Lymph nodes become inflamed or enlarged in various conditions, which can range from throat infections to life-threatening diseases such as cancers.

In another more preferred embodiment of the method(s) of the invention, the sample is a tissue sample, a blood sample or lymph.

In a further more preferred embodiment of the method(s) of the invention, the presence or amount of the biomarkers is analyzed by methods determining genetic or epigenetic modifications or transcriptional or protein levels or a combination thereof.

Methods for determining genetic or epigenetic modifications or transcriptional or protein levels have been defined herein above.

In another more preferred embodiment of the method(s) of the invention, the presence or amount of the biomarkers is determined by immunohistochemistry, mass spectrometry, Western Blot, Northern Blot, PCR, RNA in situ hybridisation or a combination thereof.

All of the above methods are well known in the art. Preferably, immunohistochemical methods include, without being limiting, tissue microarrays (TMA) as described in the appended examples. Preferably, when employing TMAs, analysis is carried out in two representative areas per TMA-spot, wherein each area comprises about 100 cells, such as for example exactly 100 cells.

In a further more preferred embodiment of the method(s) of the invention, the biomarker is protein.

The present invention further relates to a kit for predicting the course of disease in a patient having a malignant melanoma, the kit comprising: (a) means for determining the presence or amount of the set of biomarkers as defined in accordance with the methods of the present invention in a sample obtained from said malignant melanoma, and (b) instructions how to use the kit.

In its broadest sense, the term “kit” does not require the presence of any other compounds, vials, containers and the like. Preferably, the various components of the kit may be packaged in one or more containers such as one or more vials. Consequently, the various components of the kit may be present in isolation or combination. The containers or vials may, in addition to the components, comprise preservatives or buffers for storage.

“Means for determining the presence or amount of [ . . . ] biomarkers” are well known in the art and include, without being limiting, antibodies specifically binding (i.e. without cross-reacting with unrelated markers) to the biomarkers in accordance with the present invention; nucleic acid probes for the detection of the biomarkers on the nucleic acid level, such as for example nucleic acid probes specifically hybridising with parts or full-length nucleic acid molecules (DNA as well as RNA) encoding said biomarkers; sequencing primers for the analysis and detection of specific sequences of the DNA encoding the biomarkers, e.g. sequences containing mutations known to interfere with the expression of said biomarkers; amplification primers for amplifying transcribed nucleic acid molecules of the respective biomarkers; primers specific for methylated DNA for use in quantitative methylation-specific PCR (Q-MSP) (as described e.g. in Current Protocols in Human Genetics, DOI: 10.1002/0471142905.hg1006s61); and also methylation-sensitive restriction enzymes.

The term “comprising” in the context of the kit(s) of the invention denotes that further components can be present in the kit. Non-limiting examples of such further components include, as mentioned, preservatives, buffers for storage, enzymes etc.

Also encompassed by this embodiment is that the kit comprises further means for determining the presence or amount of biomarkers or reference markers different from the biomarkers of the present invention. Such biomarkers or reference markers different from the biomarkers of the present invention include, without being limiting, additional tumour markers for malignant melanoma, such as for example protein S100, HMB45, Melan-A, anti-Pan Melanoma antibodies as well as reference markers, including, without being limiting, GAPDH, RPLP0, PGK1, HSP90AB1, cyclophilin, actin.

If the kit comprises such additional means for determining the presence or amount of biomarkers or reference markers different from the markers in accordance with the present invention, it is preferred that at most 10.000 such additional markers are comprised in the kit of the invention. More preferably, at most 5.000, such as for example at most 2.000 and more preferably at most 1.000 additional nucleic markers are comprised in the kit of the invention. More preferably, at most 800, such as for example at most 600, more preferable at most 400, such as for example at most 300, at most 200, at most 100 and more preferably at most 80 additional markers are comprised in the kit of the invention. Even more preferably, at most 50, such as for example at most 40, more preferable at most 30, such as for example at most 20, at most 10, at most 9, at most 8, at most 7, at most 6, at most 5, at most 4, at most 3, at most 2 and yet more preferably at most 1 additional marker(s) is/are comprised in the kit of the invention. Also preferred is that the kit of the invention only comprises means for determining the presence or amount of the set of biomarkers as defined in accordance with the methods of the present invention.

Furthermore, the present invention also relates to a kit for deriving a treatment regimen for an individual patient having a malignant melanoma, the kit comprising: (a) means for determining the presence or amount of the set of biomarkers as defined in accordance with the present invention in a sample obtained from said malignant melanoma, (b) instructions how to use the kit.

The definitions as well as the preferred embodiments provided herein above with regard to the kit for predicting the course of disease apply mutatis mutandis also to this embodiments relating to a kit for preparing a tailored pharmaceutical composition as outlined above.

The present invention also relates to a pharmaceutical composition for use in treating or preventing malignant melanoma, wherein the pharmaceutical composition comprises (an) inhibitor(s) of CD20, Cox-2 and/or PTEN and/or (an) agent(s) affecting MTAP signalling pathways.

The term “inhibitor”, in accordance with the present invention, relates to a compound lowering the activity of a target molecule, i.e. CD20, Cox-2 and/or PTEN. The inhibitor may act preferably by performing one or more of the following effects: (i) the transcription of the gene encoding the protein to be inhibited is lowered, (ii) the translation of the mRNA encoding the protein to be inhibited is lowered, (iii) the protein performs its biochemical function with lowered or abolished efficiency in presence of the inhibitor, and (iv) the protein performs its cellular function with lowered or abolished efficiency in presence of the inhibitor.

Compounds falling in class (i) include compounds interfering with the transcriptional machinery and/or its interaction with the promoter of said gene and/or with expression control elements remote from the promoter such as enhancers but also with epigenetic control mechanisms, thus altering for example the methylation status of the promoter of a target gene. Compounds of class (ii) comprise antisense constructs and constructs for performing RNA interference (e.g. siRNA, shRNA, miRNA) well known in the art (see, e.g. Zamore (2001) Nat. Struct. Biol. 8(9), 746; Tuschl (2001) Chembiochem. 2(4), 239). Compounds of class (iii) interfere with molecular function of the protein to be inhibited, in the present case with the molecular function of CD20, Cox-2 and/or PTEN as described herein above. Accordingly, active site binding compounds are envisaged. Class (iv) includes compounds which do not necessarily bind directly to the target proteins, but still interfere with their activity, for example by binding to and/or inhibiting the function or expression of members of a pathway which comprises the target proteins. These members may be either upstream or downstream of the target protein within said pathway.

In a preferred embodiment, the level of activity (including, as defined above, the level expression) is less than 90%, more preferred less than 80%, less than 70%, less than 60% or less than 50% of the activity in the absence of the inhibitor. Yet more preferred are inhibitors lowering the level to less than 25%, less than 10%, less than 5% or less than 1% of the activity in the absence of the inhibitor.

The efficiency of the inhibitor can be quantified by comparing the level of activity in the presence of the inhibitor to that in the absence of the inhibitor. For example, as an activity measure may be used: the change in amount of mRNA formed, the change in amount of protein formed, the change in amount of activity of CD20, Cox-2 and/or PTEN, and/or a change in the cellular phenotype or in the phenotype of an organism.

The function of any of the inhibitors referred to in the present invention may be identified and/or verified by using high throughput screening assays (HTS). High-throughput assays, independently of being biochemical, cellular or other assays, generally may be performed in wells of microtiter plates, wherein each plate may contain, for example 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to the observed biological activity.

Furthermore, the determination of binding of potential inhibitors can be effected in, for example, any binding assay, preferably biophysical binding assay, which may be used to identify binding test molecules prior to performing the functional/activity assay with the inhibitor. Suitable biophysical binding assays are known in the art and comprise fluorescence polarisation (FP) assay, fluorescence resonance energy transfer (FRET) assay and surface plasmon resonance (SPR) assay.

In cases where the inhibitor acts by affecting the expression level of the target protein, the determination of the expression level of the protein can, for example, be carried out on the nucleic acid level or on the amino acid level, as described herein above.

In a preferred embodiment, the inhibitor is an antibody, an aptamer, an siRNA, an shRNA, an miRNA, a ribozyme, an antisense nucleic acid molecule or a small molecule.

The term “agents affecting MTAP signalling pathways”, in accordance with the present invention, relates to agents which do not necessarily bind directly to MTAP, but interfere with MTAP signalling activity, for example by binding to and/or inhibiting the function or expression of members of the MTAP pathway. For example, Wild et al. 2006 describe that MTAP expression correlates with responsiveness to interferon therapy, thus rendering interferon a suitable therapeutic agent in malignant melanomas expressing MTAP. Further details have been described e.g. in Behmann et al. 2003.

Inhibitors of CD20, Cox-2 and/or agents affecting MTAP signalling pathways and/or the oncogenic pathway addiction of PTEN are well known in the art and include, without being limiting, any of the compounds recited herein above.

In a more preferred embodiment, the pharmaceutical composition comprises Rituximab, Celecoxib or interferon alpha.

Rituximab is an antibody also sold under the trade names MabThera® (by Roche) or Rituxan®, (by Biogen Idec/Genentech) that has the DrugBank Accession number DB00073 and the ATC code (Anatomical Therapeutic Chemical Classification System) L01XC02.

Celecoxib, also known as Celebrex, Celebra or Onsenal (sold by Pfizer), is a sulfa non-steroidal anti-inflammatory drug and has the DrugBank Accession number APRD00373 as well as the ATC code L01XX33 M01AH01. Celecoxib has the structural formula:

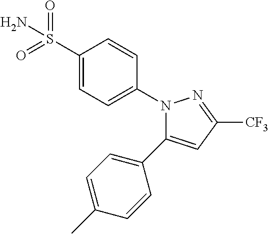 [SMILES: Cc1ccc(-c2cc(C(F)(F)F)nn2-c2ccc(S(N)(=O)=O)cc2)cc1]

Unless otherwise defined, all technical and scientific terms used herein have the same meaning as commonly understood by one of ordinary skill in the art to which this invention belongs. In case of conflict, the patent specification, including definitions, will prevail.

The figures show:

FIG. 1: The Seven-Marker Signature and Survival of 362 Patients with Primary malignant melanoma. Panel A shows the IHC expression profiles of 362 tumour specimens from the primary cohort ordered by their predicted risk score. Each column represents an individual patient consisting of the expression values of the seven-marker signature (5 risk markers and 2 protective markers). The magnitude of the corresponding risk score is plotted below for 181 low risk patients (light grey; left hand side of the Expression profile) and 181 high risk patients (dark grey; right hand side of the Expression profile). IHC expression values were scaled between 0 (light grey) and 1 (dark grey) for plotting only. White cells represent missing values (n.a.). Panels B-E show Kaplan-Meier estimates of overall and recurrence-free survival for high risk patients and low risk patients from the primary cohort according to the nine-marker signature (Panels B, C) and its reduced version, the seven-marker signature (Panels D, E), respectively. Equality in survival expectance of the subgroups is assessed by the log-rank test. Removing the two “less specific” markers (MLH1 and CD49d) from the signature does not reduce the statistical power of the predicted risk score. The difference between high risk patients and low risk patients is highly significant (p<0.001) for the seven-marker signature.

FIG. 2: Validation of the Seven-Marker Signature and the FDR Marker Selection Procedure. Kaplan-Meier estimates of overall (Panel A) and recurrence-free survival (Panel B) for the independent external test cohort of 225 patients (TMA 2) confirm the predictive prognostic power of the signature (p<0.001). In addition, the FDR marker selection procedure was tested by a 10-fold cross validation experiment on the 362 patients of the primary cohort (TMA 1) resulting in still significant estimates for overall survival (p<0.001; Panel C) and recurrence-free survival (p=0.013; Panel D).

FIG. 3: The Seven-Marker Signature and Survival of Patients with a Tumour Thickness≦2.0 mm. Kaplan-Meier estimates show a significantly lower overall (p=0.0053, Panel A) and recurrence-free survival (p=0.008, Panel B) for patients with a comparatively low tumour thickness≦2.0 mm but high-risk score. C, D. Leave-One-Out Cross Validation. To investigate the generalization error of the models produced by the FDR signature learning procedure a leave-one-out cross validation experiment was conducted on the primary cohort of 362 MM patients. The resulting risk score could significantly (p<0.001) differentiate between patients with higher or lower overall survival expectance. The two patient groups also significantly (p=0.0057) differ in recurrence-free survival. E. Permutation Test. In addition to the cross validation experiments a permutation test was conducted to assess if the signature learning procedure is over fitting the data set. The resulting signature, which was learned on permuted overall survival data, was not able (p=1) to discriminate between patients with differing survival expectance. This result indicates that the proposed learning procedure does not over fit the data. F. Coefficients and Confidence Intervals of the Seven-Marker Signature. The coefficients from the univariate Cox proportional hazard models are used in a weighted linear combination to predict the risk score for each patient. Markers with negative coefficients represent protective markers (MTAP, β-Catenin); those with positive coefficients risk markers (Bax, CD20, Bcl-X, PTEN and COX-2).

FIG. 4. Hazard Ratios of the Nine-Marker Signature learned by the FDR selection procedure. Markers with a hazard ratio smaller than 1.00 represent protective markers (MTAP, β-Catenin). Those with hazard ratios larger than 1.00 represent risk markers (Bax, Bcl-X, CD20, CD49d, COX-2, MLH1 and PTEN).

FIG. 5. Correlation between markers of the invention

FIG. 6: The Six-Marker Signature and Survival of 362 Patients with Primary Malignant Melanoma.

FIGS. 6A and B show Kaplan-Meier estimates of overall (FIG. 6A) and recurrence-free (FIG. 6B) survival for high-risk patients and low-risk patients from the primary cohort according to a further reduced six-marker signature. CD20 was removed from the seven-marker signature and the statistical power of the corresponding six-marker risk score (by Bax, Bcl-X, 3-Catenin, COX-2, MTAP, PTEN) was tested. Among the 362 patients of the primary cohort, patients with a high-risk six-marker signature had a significantly shorter median overall (FIG. 6A) and recurrence-free survival (FIG. 6B) than the patients with a low-risk six-marker signature. The difference between the two patient groups was highly significant for overall survival (p=0.000000047, FIG. 6A) and recurrence-free survival (p=0.0013, FIG. 6B), respectively. This observation supports the strong statistical power and predictive value of this set of biomarkers with the course of melanoma disease. FIG. 6 refers to the primary cohort of patients characterized in FIG. 1.

FIG. 7: The Five-Marker Signature and Survival of 362 Patients with Primary Malignant Melanoma. FIGS. 7A and B show Kaplan-Meier estimates of overall (FIG. 7A) and recurrence-free (FIG. 7B) survival for high-risk patients and low-risk patients from the primary cohort according to a further reduced five-marker signature. CD20 and PTEN were removed from the seven-marker signature and the statistical power of the corresponding five-marker risk score (by Bax, Bcl-X, β-Catenin, COX-2, MTAP) was tested. Among the 362 patients of the primary cohort, patients with a high-risk five-marker signature had a significantly shorter median overall (FIG. 7A) and recurrence-free survival (FIG. 7B) than the patients with a low-risk five-marker signature. The difference between the two patient groups was highly significant for overall survival (p=0.00000066, FIG. 7A) and recurrence-free survival (p=0.0024, FIG. 7B), respectively. This observation supports the strong statistical power and predictive value of this set of only five biomarkers with the course of melanoma disease. FIG. 7 also refers to the primary cohort of patients characterized in FIG. 1.

FIG. 8: Immunohistochemically stained TMA Specimens illustrating the Seven-Marker Signature for one Patient with High-Risk and one Patient with Low-Risk Melanoma. The low-risk melanoma (Column C) showed a strong cytoplasmic staining for β-Catenin and MTAP, respectively. Immunoreactivity of these two protective markers was not found in the high-risk melanoma (Column D). In contrast, the high-risk melanoma demonstrated a moderate to strong cytoplasmic staining for Bax, CD20, Bcl-X, PTEN and COX-2.

The examples illustrate the invention:

### EXAMPLE 1

### Materials and Methods

### Tissue Microarrays (TMAs)

TMAs were constructed as described previously (Alonso, S. R. et al., 2004; Wild, P. J. et al., 2006; Meyer, S. et al., 2009) and based on primary melanoma material, collected between 1994 and 2006. TMA 1, the primary cohort, contained tissue punch samples from 364 consecutive (non-selected), formalin-fixed, paraffin-embedded malignant melanoma of 364 different patients and were from the Department of Dermatology, University Hospital of Regensburg, Germany. TMA 2, the secondary cohort, which was used as independent external validation cohort, consisted of consecutive (non-selected) melanoma samples from 235 patients of the Department of Dermatology, University Hospital Hamburg-Eppendorf, Germany. For patients with multiple subsequent neoplasms, only initial and single primary malignant melanomas were included. H&E-stained slides of all malignant melanomas were evaluated by two histopathologists. The clinico-pathological characteristics of the two independent cohorts of melanoma patients are given in Table 2. Clinical follow-up data, provided by the local tumour registries, were available for all patients of the primary cohort (n=364) and 231 patients of the secondary cohort. Patients were censored at 120 months, if their follow-up exceeded the 10-year scope of the study. The study for both cohorts was approved by the local scientific ethics committees (approvals no.: 07/093 for Regensburg and MC-028/08 for Hamburg). The retrospective study was conducted according to the Declaration of Helsinki Principles.

TABLE 2
Characterization and Comparison of the Primary Cohort
(TMA 1) and the External Test Cohort (TMA 2).

| Primary Cohort | | Ext. Test cohort | |
|---|---|---|---|
| Origin | | | |
| Regensburg | | Hamburg | |
| N | % | N | % |

TMA characteristics

| | | | | |
|---|---|---|---|---|
| No. of patients | 364 | | 235 | |
| No. follow-up | 364 | 100 | 231 | 98.3 |
| No. of patients with at | 362 | 99.5 | 225 | 95.7 |
| least 1 signature marker | | | | |

TMA Spots

| | | | | |
|---|---|---|---|---|
| No. of biomarkers | 70 | | 7 | |
| Valid spots | 23106 | 90.7 | 1541 | 93.7 |
| Missing spots | 2374 | 9.3 | 104 | 6.3 |

Clinicopathological characteristics

| Age | | | | | |
|---|---|---|---|---|---|
| <=60 | | 180 | 49.5 | 139 | 59.1 |
| >60 | | 184 | 50.5 | 92 | 39.1 |
| unknown | | | | 4 | 1.7 |
| Sex | | | | | |
| Male | | 195 | 53.6 | 126 | 53.6 |
| Female | | 169 | 46.4 | 105 | 44.7 |
| unknown | | | | 4 | 1.7 |
| Tumor thickness | | | | | |
| <=1 | mm | 163 | 44.8 | 110 | 46.8 |
| 1.01--2 | mm | 92 | 25.3 | 47 | 20 |
| 2.01--4 | mm | 61 | 16.8 | 36 | 15.3 |
| >4 | mm | 42 | 11.5 | 36 | 15.3 |

| | | | | |
|---|---|---|---|---|
| unknown | 6 | 1.6 | 6 | 2.6 |
| Clark level | | | | |
| 1 | 2 | 0.5 | 1 | 0.4 |
| 2 | 75 | 20.6 | 39 | 16.6 |
| 3 | 106 | 29.1 | 80 | 34 |
| 4 | 149 | 40.9 | 90 | 38.3 |
| 5 | 14 | 3.8 | 19 | 8.1 |
| unknown | 18 | 4.9 | 6 | 2.6 |
| Growth pattern | | | | |
| SSM | 170 | 46.7 | 146 | 62.1 |
| NMM | 56 | 15.4 | 49 | 20.9 |
| LMM | 42 | 11.5 | 12 | 5.1 |
| ALM | 28 | 7.7 | 8 | 3.4 |
| NOS | 68 | 18.7 | 20 | 8.5 |

Immunohistochemical data

| | | | | |
|---|---|---|---|---|
| Bax | | | | |
| 0 | 5 | 1.4 | 5 | 2.1 |
| 1 | 60 | 16.5 | 49 | 20.9 |
| 2 | 95 | 26.1 | 81 | 34.5 |
| 3 | 88 | 24.2 | 56 | 23.8 |
| 4 | 88 | 24.2 | 29 | 12.3 |
| unknown | 28 | 7.7 | 15 | 6.4 |
| b-Catenin | | | | |
| 0 | 10 | 2.7 | 4 | 1.7 |
| 1 | 121 | 33.2 | 43 | 18.3 |
| 2 | 106 | 29.1 | 108 | 46 |
| 3 | 71 | 19.5 | 53 | 22.6 |
| 4 | 17 | 4.7 | 11 | 4.7 |
| unknown | 39 | 10.7 | 16 | 6.8 |
| CD20 | | | | |
| 0 | 333 | 91.5 | 154 | 65.5 |
| 1 | 12 | 3.3 | 48 | 20.4 |
| 2 | 4 | 1.1 | 16 | 6.8 |
| 3 | 1 | 0.3 | 1 | 0.4 |
| unknown | 14 | 3.8 | 16 | 6.8 |
| BCL-X | | | | |
| 0 | 151 | 41.5 | 66 | 28.1 |
| 1 | 167 | 45.9 | 117 | 49.8 |
| 2 | 26 | 7.1 | 38 | 16.2 |
| 3 | 1 | 0.3 | 4 | 1.7 |
| unknown | 19 | 5.2 | 10 | 4.3 |
| MTAP | | | | |
| 0 | 56 | 15.4 | 103 | 43.8 |
| 1 | 245 | 67.3 | 101 | 43 |
| 2 | | | 16 | 6.8 |
| unknown | 63 | 17.3 | 15 | 6.4 |
| PTEN | | | | |
| 0 | 57 | 15.7 | 23 | 9.8 |
| 1 | 140 | 38.5 | 87 | 37 |
| 2 | 116 | 31.9 | 76 | 32.3 |
| 3 | 28 | 7.7 | 25 | 10.6 |
| 4 | 4 | 1.1 | 8 | 3.4 |
| unknown | 19 | 5.2 | 16 | 6.8 |
| Cox-2 | | | | |
| 0 | 121 | 33.2 | 20 | 8.5 |
| 1 | 188 | 51.6 | 103 | 43.8 |
| 2 | 39 | 10.7 | 73 | 31.1 |
| 3 | 5 | 1.4 | 21 | 8.9 |
| 4 | | | 2 | 0.9 |
| unknown | 11 | 3 | 16 | 6.8 |
| CD49d | | | | |
| 0 | 63 | 17.3 | n.a | |
| 1 | 137 | 37.6 | | |
| 2 | 78 | 21.4 | | |
| 3 | 33 | 9.1 | | |
| 4 | 3 | 0.8 | | |
| unknown | 50 | 13.7 | | |
| MLH1 | | | | |
| 0 | 65 | 17.9 | n.a | |
| 1 | 130 | 35.7 | | |
| 2 | 99 | 27.2 | | |
| 3 | 37 | 10.2 | | |
| 4 | 13 | 3.6 | | |
| unknown | 20 | 5.5 | | |

Reported are the number of counts and the associated percentages for all specimens on the tissue microarrays. CD49d and MLH1 are not contained in the final seven-marker signature and therefore were not analyzed on the external test TMA 2. Missing values are listed as “unknown”.

### Immunohistochemical Analysis

Paraffin-embedded preparations of melanoma tissues were screened for protein expression according to standardized immunohistochemical (IHC) protocols as described previously (Alonso, S. R. et al., 2004; Wild, P. J. et al., 2006; Meyer, S. et al., 2009). The primary antibodies used in this study were selected for reporting on key aspects of apoptosis, cell cycle, signal transduction, cell adhesion, melanoma differentiation and proliferation, and tumour metabolism.

All IHC investigations were based on an avidin-biotin peroxidase method with a 3-amino-9-ethylcarbazole (AEC) chromatogen. After antigen retrieval (steam boiler with citrate-buffer, pH 6.0 or with Tris-EDTA-buffer, pH 9.0 for 20 min), immunohistochemistry was carried out applying the ZytoChemPlus HRP Broad Spectrum Kit (Zytomed Systems, Berlin, Germany) according to the manufacturer's instructions. IHC stainings were performed for 70 different primary antibodies (source and concentration are listed in Table 3). Cytoplasmic and nuclear markers were visualized with AEC solution (AEC+High Sensitivity Substrate Chromogen, ready-to-use, DAKO, Glostrup, Denmark). The red colour of the AEC substrate chromogen (3-amino-9-ethylcarbazole) is very beneficial to rule out the possibility of a role of endogenous melanin in the observed reactivity. All sections were counterstained with hematoxylin (DAKO). Negative controls were obtained by omitting the primary antibody. Two dermatohistopathologists performed a blinded evaluation of the stained slides without knowledge of clinical data. The specificity of the commercial antibodies has been thoroughly tested by Western blotting using melanocytes and a variety of human cell lines including several melanoma cell lines.

TABLE 3
Properties of the 70 Biomarker Candidates for Malignant
Melanoma Immunohistochemically analyzed in this Study

| | HUGO: | | | | | | |
|---|---|---|---|---|---|---|---|
| Protein | Approved | Functional | | | | Cellular | Positive |
| Name | Symbol | Group | Source | Clone | Dilution | Localization | Control |
| Akt3 | AKT3 | Signaling, | Abgent | Rabbit, | 1:100 | Cytoplasmic | Breast |
| | | Apoptosis | | Polyclonal | | | cancer |
| Phospho-Akt | Phospho- | Signaling, | Cell | Rabbit, | 1:10 | Cytoplasmic | Breast |
| (Thr308) | AKT3 | Apoptosis | Signaling | 244F9H2 | | | cancer |
| Bax | BAX | Apoptosis | Cell | Rabbit, | 1:10 | Cytoplasmic | Lung |
| | | | Signaling | polyclonal | | | cancer |
| Bcl2 | BCL2 | Apoptosis | Dako | Mouse, | 1:100 | Cytoplasmic | Kidney |
| | | | | 124 | | | |
| Bcl-X | BCL2L1 | Apoptosis | Diagnostic | Mouse, | 1:10 | Cytoplasmic, | Tonsil |
| | | | Biosystems | 2H12 | | cell membrane | |
| Bcl2L1 | BCL2L1 | Apoptosis | Abcam | Mouse, | 1:200 | perinuclear | Melanoma |
| | | | | SPM165 | | | |
| BMI1 | BMI1 | Transciption | Abgent | Rabbit, | 1:25 | Cytoplasmic | Breast |
| | | | | polyclonal | | | cancer |
| B-Raf | BRAF | Signaling, | Epitomics | Rabbit, | 1:50 | Cytoplasmic | Prostate |
| | | Apoptosis | | EP152Y | | | cancer |
| E-Cadherin | CDH1 | Cell-cell contact | Chemicon | Mouse, | r-t-u | Cell membrane | Breast |
| | | | | 36B5 | | | cancer |
| P-Cadherin | CDH3 | Cell-cell contact | BD | Mouse, | 1:20 | Cell membrane | Placenta |
| | | | Biosciences | 56 | | | |
| β-Catenin | CTNNB1 | Cell-cell contact | Cell | Rabbit, | 1:250 | Cytoplasmic, | Breast |
| | | | Siganling | polyclonal | | nuclear, | cancer |
| | | | | | | cell membrane | |
| Phospho-β- | Phospho- | Cell-cell contact | Cell | Rabbit, | 1:50 | Nuclear | Breast |
| Catenin | CTNNB1 | | Signaling | polyclonal | | | cancer |
| Caveolin | CAV1 | Cell-cell contact | BD | Rabbit, | 1:1000 | Cell membrane | Placenta |
| | | | Biosciences | polyclonal | | | |
| CD 20 | MS4A1 | Differentiation, | Zytomed | Mouse, | r-t-u | Cell membrane | Tonsil |
| | | Stem cell marker | | L26 | | cytoplasmic | |
| | | cand. | | | | | |
| CD 44 | CD44 | Cell-cell contact, | Lab Vision | Mouse, | 1:2000 | Cell membrane | Tonsil |
| | | Stem cell marker | | 156-3C11 | | | |
| | | cand. | | | | | |
| CD 49d | ITGA4 | Cell-cell contact, | Acris | Rabbit, | 1:50 | Cell membrane | Breast |
| | | Stem cell marker | | polyclonal | | | cancer |
| | | cand. | | | | | |
| CD 117 | KIT | Proliferation, | Dako | Rabbit, | 1:600 | Cell membrane | Colorectal |
| (c-kit) | | Stem cell marker | | polyclonal | | | cancer |
| | | cand. | | | | | |
| CD 166 | ALCAM | Cell-cell contact, | Abcam | Mouse, | 1:50 | Cell membrane | Prostate |
| | | Stem cell marker | | MOG/07 | | | cancer |
| | | cand. | | | | | |
| CD 171 | L1CAM | Cell-cell contact | Sigma | Rabbit | 1:350 | Cytoplasmic | Breast |
| | | | | | | | cancer |
| CDK2 | CDK2 | Cell cycle | Thermo | Mouse, | 1:200 | Cytoplasmic, | Tonsil |
| | | | Scientific | 2B6 + 8D4 | | nuclear | |
| c-Myc | MYC | Cell cycle, | Santa Cruz | Mouse, | 1:500 | Cytoplasmic, | Colorectal |
| | | Proliferation | | monoclonal | | nuclear | cancer |
| Cox-2 | MT-CO2 | Metabolism | Cayman | Mouse, | 1:200 | Cytoplasmic | Colorectal |
| | | | | monoclonal | | | cancer |
| CXCR4 | CXCR4 | Cell-cell contact, | Acris | Rabbit, | 1:750 | Cytoplasmic, | Breast |
| | | Stem cell marker | | polyclonal | | cell membrane | cancer |
| | | cand. | | | | | |
| Cyclin A | CCNA1 | Cell cycle | Novo Castra | Mouse, | 1:50 | nuclear | Tonsil |
| | | | | 6E6 | | | |
| Cyclin D1 | CCND1 | Cell cycle | Novo Castra | Mouse, | 1:20 | Nuclear, | Breast |
| | | | | DCS-6 | | cytoplasmic | cancer |
| Eph B2 | EPHB2 | Cell-cell contact | Abcam | Rabbit, | 1:100 | Cell membrane | Breast |
| | | | | polyclonal | | cytoplasmic | cancer |
| Ezrin | EZR | Signaling | Abcam | Mouse, | 1:100 | Cell membrane | Lung |
| | | | | 3C12 | | | Cancer |
| Ezrin | EZR | Signaling | Abcam | Mouse, | 1:100 | Cell membrane | Lung |
| | | | | 3C12 | | | Cancer |
| Fas | FAS | Apoptosis | Cell | Rabbit, | 1:10 | Cytoplasmic | Colorectal |
| | | | Signaling | C18C12 | | | cancer |
| FZD-7 | FZD7 | Signaling | Acris | Rabbit, | 1:250 | Cytoplasmic | Placenta |
| | | | | polyclonal | | | |
| Glut-1 | SLC2A1 | Metabolism | Acris | Mouse, | 1:200 | Cell membrane | Breast |
| | | | | SPM498 | | | cancer |
| HIF-1α | HIF1A | Metabolism | R + D | Mouse, | 1:20 | Cytoplasmic, | Breast |
| | | | | 241812 | | nuclear | cancer |
| Anti-Melanosome | −/− | Differentiation | Dako | Mouse, | 1:50 | Cytoplasmic | Melanoma |
| HMB45 | | | | HMB45 | | | |
| IGF-2 | IGF2 | Proliferation | Abcam | Rabbit, | 1:200 | Cytoplasmic | Placenta |
| | | | | polyclonal | | | |
| iNOS | ISYNA1 | Metabolism | Abcam | Rabbit, | 1:200 | Cytoplasmic | Lung |
| | | | | polyclonal | | | cancer |
| Ki67 | MKI67 | Cell cycle, | Dako | Mouse, | 1:100 | Nuclear | Skin |
| | | Proliferation | | MIB-1 | | | |
| MHC 1 | MYH11 | Cell-cell contact | Abcam | Mouse, | 1:100 | Cell membrane | Kidney |
| | | | | 3F8 | | | |
| Melan A | MLANA | Differentiation | Novo Castra | Mouse, | 1:50 | Cytoplasmic | Skin |
| | | | | A103 | | | |
| MITF | MITF | Differentiation | Dako | Mouse, | 1:50 | Nuclear | Melanoma |
| | | | | D5 | | | |
| MLH 1 | MLH1 | DNA repair | BD | Mouse, | 1:50 | Nuclear, | Colorectal |
| | | | Pharmingen | G168-15 | | cytoplasmic | cancer |
| MSH2 | MSH2 | DNA repair | Calbiochem | Mouse, | 1:40 | Nuclear, | Colorectal |
| | | | | FE11 | | cytoplasmic | cancer |
| MTAP | MTAP | Metabolism | Protein Tech | Rabbit, | 1:500 | Cytoplasmic | Breast |
| | | | Group | polyclonal | | | cancer |
| MTSS1 | MTSS1 | Cytoskeletal | Abnova | Mouse, | 1:500 | Nuclear, | Colorectal |
| | | remodeling | | 2G9 | | cytoplasmic | cancer |
| MUM1p | IRF4 | Transcription | Santa Cruz | Mouse, | 1:10 | Nuclear, | Breast |
| | | | | monoclonal | | cytoplasmic | cancer |
| NF-kB | RELA | Transcription | Santa Cruz | Mouse, | 1:500 | Nuclear, | Breast |
| | | | | F-6 | | cytoplasmic | cancer |
| N-Ras | NRAS | Signaling | Santa Cruz | Mouse, | 1:50 | Cytoplasmic | Lymph |
| | | | | F155 | | | node |
| N-Ras | NRAS | Signaling | Santa Cruz | Mouse, | 1:50 | Cytoplasmic | Lymph |
| | | | | F155 | | | node |
| p 14 | CDKN2A | Cell cycle | Cell Signaling | Mouse, | 1:10 | Nuclear, | Breast |
| | | | | 4C6/4 | | cytoplasmic | cancer |
| p 15 | CDKN2B | Cell cycle | Acris | Mouse, | 1:25 | Nuclear | Colorectal |
| | | | | 15P06 | | | cancer |
| p 16 | CDKN2A | Cell cycle | Santa Cruz | Mouse, | 1:50 | Nuclear | Colorectal |
| | | | | F-12 | | | cancer |
| p 21 | CDKN1A | Cell cycle | Dako | Mouse, | 1:50 | Nuclear | Colorectal |
| | | | | SX118 | | | cancer |
| p 27 | CDKN1B | Cell cycle | Dako | Mouse, | 1:50 | Nuclear | Lymph |
| | | | | SX53G8 | | | node |
| p 53 | TP53 | Cell cycle, | Dako | Mouse, | 1:25 | Nuclear, | Breast |
| | | Apoptosis | | DO-7 | | cytoplasmic | cancer |
| p 75 (NGFR) | NGFR | Differentiation | Abcam | Rabbit, | 1:100 | Cell membrane | Placenta |
| | | | | EP1039Y | | | |
| PGF | PGF | Proliferation | ProteinTech | Rabbit, | 1:50 | Cytoplasmic | Breast |
| | | | Group | polyclonal | | | cancer |
| PMP2 | PMP2 | Differentiation | ProteinTech | Rabbit, | 1:100 | Cytoplasmic | Glioma |
| | | | Group | polyclonal | | | |
| PPAR α | PPARA | Signaling | Abcam | Rabbit, | 1:500 | Cytoplasmic, | Breast |
| | | | | polyclonal | | nuclear | cancer |
| PTEN | PTEN | Signaling | Cell Signaling | Rabbit, | 1:100 | Cytoplasmic, | Colorectal |
| | | | | 138G6 | | nuclear | cancer |
| Rb | RB1 | Cell cycle | Calbiochem | Mouse, | 1:50 | Nuclear | Colorectal |
| | | | | LM95.1 | | | cancer |
| Phospho-Rb | Phospho- | Cell cycle | Cell Signaling | Rabbit, | 1:50 | Nuclear | Colorectal |
| | RB1 | | | polyclonal | | | cancer |
| Ro-52 | TRIM21 | Autoimmunology | Santa Cruz | Mouse, | 1:200 | Nuclear | n. d. |
| | | | | D-12 | | | |
| Survivin | BIRC5 | Apoptosis | Cell Signaling | Rabbit, | 1:100 | Nuclear | Colorectal |
| | | | | 71G4 | | | cancer |
| SKP2 | SKP2 | Cell cycle | Zytomed | Rabbit, | 1:200 | Nuclear, | Prostate |
| | | | | polyclonal | | cytoplasmic | cancer |
| STAT1 | STAT1 | Signaling | Cell Signaling | Rabbit, | 1:200 | Cytoplasmic, | Colorectal |
| | | | | 42H3 | | nuclear | cancer |
| Phospho- | | Signaling | Abcam | Rabbit, | 1;100 | Cytoplasmic, | Breast |
| STAT1(S727) | | | | polyclonal | | nuclear | cancer |
| S1P1 | S1PR1 | Cell cycle | Cayman | Rabbit, | 1:50 | Cytoplasmic | Breast |
| | | | | polyclonal | | | cancer |
| TGF-β1 | TGFB1 | Proliferation | Zytomed | Rabbit, | 1:25 | Cytoplasmic, | Colorectal |
| | | | | polyclonal | | cell membrane | cancer |
| Topoisomerase | TOP2A | DNA repair | Dako | Mouse, | 1:100 | Nuclear, | Placenta |
| llα | | | | Ki-S1 | | cytoplasmic | |
| VEGFR-2 | KDR | Proliferation | Cell Signaling | Rabbit, | 1:200 | Cytoplasmic | Colorectal |
| | | | | 55B11 | | | cancer |
| XIAP (Birc4) | XIAP | Apoptosis | Lifespan | Rabbit, | 1:1000 | Cytoplasmic | Placenta |
| | | | | polyclonal | | | |

antiibodies investigated are listed indicating source, dilution, pattern of reactivity and positive control. The described signature was statistically learned by the FDR selection procedure from this pool of 70 biomarkers.

A dermatohistopathologist and a surgical pathologist performed a blinded, stringent evaluation of the stained slides as previously described (Wild, P. J. et al., 2006; Meyer, S. et al., 2009). Cytoplasmic and nuclear immuno-reactivity were evaluated using a stepwise scoring system (0 to 4+): 0 (negative): no cytoplasmic staining or 0% of cell nuclei stained; 1+: weak cytoplasmic staining or less than 20% of cell nuclei stained; 2+: moderate cytoplasmic staining or 21 to 50% of cell nuclei stained; 3+: strong cytoplasmic staining or 51 to 90% of cell nuclei stained; 4+: very strong cytoplasmic staining or nuclear staining greater than 90%. This semi-quantitative scoring system was consistently used for all 70 markers analyzed. Cytoplasmic markers were estimated according to the staining intensity found in the melanoma cells of the individual TMA spot. For nuclear markers, the percentage of melanoma cell nuclei with positive staining was assessed. TMA spots with a lack of tumour tissue or presence of necrosis or crush artefact were excluded from the analysis.

### Statistical Analysis

An estimation of statistical power versus total sample size N for different hazard ratios was performed. Accordingly, the available sample size of 364 analyzable patients on TMA1 would be sufficient to detect a difference concerning survival with a significance of p<0.05 and a power of almost 100%. Calculations were performed using the respective models of the PASS 2008 software (NCSS, Kaysville, Utah).

One of the main statistical problems in large scale IHC studies are missing values in the design matrix due to missing or corrupt spots on the TMA. The more markers are investigated the higher the chance that at least one value is missing per patient. Frequently, this problem is tackled by either sacrificing a larger number of patient records or by employing volatile multiple imputation techniques. In this study 9.3% of values are missing which would reduce the set of patients with all IHC measurements from 364 to 170. Algorithms like random survival forests (Ishwaran, H. et al., 2008) and ensemble learning with gradient boosting (Hothorn, T. et al., 2006) are capable of dealing with missing values, but lead to models, which are not intuitively interpretable and difficult to implement in clinical practice. To overcome these problems the following learning procedure was employed which is invariant to missing values and results in an easily interpretable and a practically applicable linear model.

Prognostic power of the 70 markers was assessed by learning univariate proportional hazard models (Cox, D. R. et al., 1972,) yielding 11 markers significantly associated with overall survival. To correct for multiple testing, the false discovery rate (FDR) procedure (Benjamini, Y. et al., 1995) was applied with a FDR of 0.15 reducing the set of significantly associated markers to 9. A risk score was calculated for each patient by a linear combination of the univariate Cox regression coefficients β and the corresponding IHC measurements x. Finally, the score is normalized by the number of markers measured:

$$score ( x ) = ( {\sum }_{i = 1}^{\vert x \vert} ( {β}_{i} {x}_{i} ) {1}_{[ ∃ {x}_{i} ]} ) \text{/} ( {\sum }_{i = 1}^{\vert x \vert} {1}_{[ ∃ {x}_{i} ]} )$$

Based on this risk score, patients were assigned to a high risk group and a low risk group, split at the 50^{th} percentile (median) of all scores. Thus, the final model consists of the coefficient vector β and the median threshold θ.

A simplified version of this risk score calculation is as described herein above, i.e.:

$$score ( x ) = ( {\sum }_{i = 1}^{D} ( {β}_{i} {x}_{i} ) {α}_{i} ) \text{/} ( {\sum }_{i = 1}^{D} {α}_{i} ) , \\ {α}_{i} = \{ \begin{matrix}1 , & if {x}_{i} exists \\ 0 , & if {x}_{i} is missing\end{matrix}$$

Nonparametric Kaplan-Meier estimators (Kaplan, E. L. et al., 1958) were used to analyze overall survival and recurrence-free survival. Differences between survival estimates were assessed with the log-rank test (LRT) (Mantel, N. et al., 1966). P-values below 0.05 were considered to indicate statistical significance. Statistical analyzes were conducted using R version 2.11 (R Development Core Team (2009). R: A language and environment for statistical computing. R Foundation for Statistical Computing, Vienna, Austria. ISBN 3-900051-07-0).

### Statistical Validation

The validity of the learning procedure and hence the accuracy of the signature was assessed in three different validation experiments. First, leave-one-out cross validation was employed by excluding one patient at a time from the training set and subsequently scoring the left out patient with the signature learned from the rest of the patients. Repeating this procedure 364 times yields a leave-one-out score estimate for each patient in the study. The resulting difference between high risk patients and low risk patients was highly significant (p<0.001) and is depicted in FIG. 3C and D.

Second, 10-fold cross validation was conducted by partitioning the dataset into 10 parts of equal size using 90% of the patients for learning and 10% for validation. The procedure was repeated 10 times resulting in a 10-fold score for each patient. As expected, the resulting differentiation between high risk and low risk patient was worse in terms of the LRT p-value but was still highly significant (p<0.001) as shown in FIG. 2C and D.

The third validation experiment was conducted to assess if the proposed marker selection procedure is prone to over fitting. To this end, the target variable was randomly permuted and a model was learned to predict the risk score based on this distorted data. FIG. 3E illustrates that it was impossible to learn a meaningful score (p>0.5) based on the permuted labels. Although a large number of markers were analyzed to learn the signature, this result indicates that the proposed algorithm does not over fit.

### EXAMPLE 2

### The Nine-Marker Signature and Survival

The proposed learning procedure based on the Cox regression coefficients and multiple testing correction with FDR yielded nine markers which were correlated with death from any cause. Two of these markers were protective markers (associated with a hazard ratio of less than 1.00) and seven were risk markers (associated with a hazard ratio of more than 1.00) (FIG. 4).

Among the nine markers were Bax and Bcl-X, two major regulators of the “intrinsic” mitochondrial apoptosis pathway (Lowe, S. W. et al., 2004). Moreover, β-Catenin, a key downstream effector in the Wnt signaling pathway (Delmas, V et al., 2007,) and, CD20, a known B-cell marker recently suggested as candidate marker for melanoma stem cells (Zabierowski, S. E. et al., 2008). CD49d, an α4-integrin (ITGA4) participating in cell-surface mediated signaling and adhesion, was included, too (Kuphal, S. et al., 2005). Apart from this, COX-2, a cyclooxygenase also referred to as Prostaglandin H Synthase 2 with overexpression in a variety of tumors including melanoma tumors was part of the signature (Meyer, S. et al., 2009). Two other markers were MLH1, a DNA mismatch repair protein (Korabiowska, M. et al., 2006,) and MTAP, a “housekeeping enzyme” in polyamine metabolism and modulating protein of interferon response mechanisms (Wild, P. J. et al., 2006; Behrmann, I. et al., 2003). Finally, the tumor suppressor phosphatase and tensin homolog PTEN was identified as another signature protein. PTEN counteracts one of the most critical cancer promoting pathways (Zhang, S. et al., 2010,) the phosphatidylinositol 3-kinase (PI3K)/Akt signaling pathway. Clinically, PTEN mutations and deficiencies are prevalent in many types of human cancers and loss of functional PTEN has substantial impact on multiple aspects of cancer development. MTAP and β-Catenin were the only protective markers, whereas the other seven markers (Bax, Bcl-X, CD20, CD49d, COX-2, MLH1, PTEN) were assigned risk markers.

Table 1 lists the characteristics of 362 patients in the study (two patients were removed due to lack of all nine markers from the signature). Among these 362 patients of the primary cohort tumors with high risk scores expressed risk markers, whereas tumors with low risk scores expressed protective markers (FIG. 1A). Patients with a high-risk nine-marker signature had a lower median overall survival than those with a low-risk nine-marker signature (90 months versus not reached) (FIG. 1B). Patients with tumors with a high-risk marker signature were associated with a lower median recurrence-free survival than patients with tumors with a low-risk gene signature (36 months versus 88) (FIG. 1C).

The cross validation experiments showed comparable results and demonstrated that learning a marker signature for overall survival is feasible and reproducible (FIG. 2C, D). For leave-one-out cross validation, patients with high risk scores had a median survival of 94 month whereas median survival for patients with low risk signature was not reached (FIG. 3C). The difference in survival expectance between patients with high-risk score and low-risk score was highly significant (p=0.000067). Although 10-fold cross validation has lower bias and higher variance the difference between the high risk and low risk group (94 month versus not reached) was still significant (p=0.00017) as shown in FIG. 2C. In contrast to the cross validation experiments it was not possible to learn a signature to predict permuted labels (p>0.5), which indicates that the proposed learning procedure is not over fitting. In the permutation test median survival was not reached by any risk-group (FIG. 3E).

### EXAMPLE 3

### The Seven-Marker Signature and Survival

The aim of this study was to provide a maximum of prognostic and therapeutically relevant information by a minimum of markers combined in a clear signature. For the sake of clinical feasibility and cost saving, an IHC marker set suitable for routine clinical assessment should be based on a limited number of antibodies. Accordingly, the 9-marker signature was reduced by the risk marker with the lowest Cox regression coefficients β, i.e. MLH1 (β=0.254). Subsequently, the remaining 8 risk markers were evaluated regarding their impact on cancer development and progression and potential therapeutic implications. In this setting, CD49d, an α4-integrin (ITGA4) participating in cell-surface mediated signaling and adhesion, was considered to be the most dispensable marker. In particular, Western blot analysis of this 70-180 kDa protein did not reveal one specific but several bands for a panel of melanoma cell lines and melanocytes, respectively. Specificity of all other IHC antibodies of the signature could be confirmed by immunoblotting.

Among the 362 patients of the primary cohort, patients with a high-risk seven-marker signature (Bax, Bcl-X, β-Catenin, CD20, COX-2, MTAP, PTEN) had a shorter median overall survival than the patients with a low-risk seven-marker signature (88 months versus not reached) and the difference between the two patient groups was highly significant (p=0.0000000042) (FIG. 1D). The high-risk seven-marker signature was associated with a median recurrence-free survival of 33 months, whereas the low-risk seven-marker signature was associated with a median recurrence-free survival of 88 months (LRT p=0.00034, FIG. 1E).

According to multivariate Cox regression analysis, the seven-marker risk score, tumor thickness, sex, and age were significantly associated with death from any cause among the 356 patients (6 observations were deleted due to missing values) (Table 1).

A subgroup analysis of 253 patients with a tumor depth of ≦2 mm revealed that those 148 patients with a high-risk marker signature had a significant (p=0.0053) shorter overall survival (FIG. 3A) and recurrence-free survival (p=0.008) than the 105 patients with a low-risk marker signature (FIG. 3B).

### EXAMPLE 4

### Validation of the Seven-Marker Signature on an External Test Cohort

The clinical characteristics of the 225 patients in the external test cohort are listed in Table 4. Patients with a high-risk marker signature had a significantly (p=0.000017) different survival expectance and shorter median overall survival compared to patients with a low-risk signature (95 months versus not reached) (FIG. 2A). According to multivariate Cox regression including sex, age, tumor thickness, ulceration and nodal status, the seven-marker signature was significantly associated with overall survival (p=0.0000098, Table 1). Additionally, the recurrence-free survival differed significantly between the two risk groups (p=0.004; FIG. 2B).

### EXAMPLE 5

### The Six-Marker Signature and Survival

After the seven-marker signature was reduced by the marker CD20, the corresponding six-marker signature (Bax, Bcl-X, β-Catenin, COX-2, MTAP, PTEN) still showed a significant correlation with overall and recurrence-free survival; i.e. among the 362 patients of the primary cohort, patients with a high-risk six-marker signature (Bax, Bcl-X, 3-Catenin, COX-2, MTAP, PTEN) had a significantly shorter median overall (FIG. 6A) and recurrence-free survival (FIG. 6B) than the patients with a low-risk six-marker signature. The difference between the two patient groups was highly significant for overall survival (p=0.000000047, FIG. 6A) and recurrence-free survival (p=0.0013, FIG. 6B), respectively.

### EXAMPLE 6

### The Five-Marker Signature and Survival

After the seven-marker signature was reduced by the markers CD20 and PTEN, the corresponding five-marker signature (Bax, Bcl-X, β-Catenin, COX-2, MTAP) still showed a significant correlation with overall and recurrence-free survival; i.e. among the 362 patients of the primary cohort, patients with a high-risk five-marker signature (Bax, Bcl-X, 3-Catenin, COX-2, MTAP) had a significantly shorter median overall (FIG. 7A) and recurrence-free survival (FIG. 7B) than the patients with a low-risk five-marker signature. The difference between the two patient groups was highly significant for overall survival (p=0.00000066, FIG. 7A) and recurrence-free survival (p=0.0024, FIG. 7B), respectively.

TABLE 4
Clinical characteristic of the External Test Cohort of Patients with MM (TMA 2)

| | High risk | Low risk | p-Value | Multivariate Cox Regression Analysis | |
|---|---|---|---|---|---|
| | (N = 181) | (N = 181) | high vs. low risk | Hazard ratio(95% CI) | p-Value |
| 7-Marker risk score | 0.270 ± 0.098 | 0.004 ± 0.071 | <<0.0001 *¹ | 24.91 (3.84-161.71) | 0.00076 *** |
| Age - yr | 55.2 ± 16 | 52.6 ± 6.6 | 0.267 *¹ | 1.02 (1.00-1.04) | 0.016 |
| Sex - no. of patients (%) | | | | | |
| Male | 81 (55.1) | 41 (54.7) | 1 *² | 0.67 (0.39-1.14) | 0.14 |
| Female | 66 (44.9) | 34 (45.3) | | | |
| Tumor thickness - mm | 2.55 ± 2.67 | 1.06 ± 1.17 | 0.0000000512 *¹ | 1.28 (1.19-1.38) | 0.000000000028 *** |

*¹ Welch two sample t-test
*² Fisher's exact test
* p-Value < 0.05
*** p-Value < 0.001

Comparing high-risk patients (first column) with low-risk patients (second column) based on their seven-marker risk score shows significant difference in tumour thickness (p<0.001) and no difference in sex (p=1) and age (p=0.267). Furthermore, hazard ratios and p-values are reported for a multivariate Cox regression model comprising all listed variables. Regarding overall survival the seven-marker risk score is statistically significant (p<0.001) independent of sex, age and tumour thickness. Continuous variables are reported with mean and standard deviation and categorical variables are listed with number of counts and percentages.

1. A method of predicting the course of disease in a patient having a malignant melanoma, the method comprising determining in a sample obtained from said patient the presence or amount of at least five biomarkers selected from the group comprising or consisting of MTAP, PTEN, Bax, Bcl-X, β-Catenin, CD20, Cox-2, CD49d and MLH1, wherein the absence or decreased amount of MTAP and β-Catenin and/or the presence or increased amount of PTEN, Bax, Bcl-X, CD20, Cox-2, CD49d and MLH1 is associated with a disadvantageous course of disease.

2. The method according to claim 1, wherein the at least five biomarkers include PTEN and/or MTAP.

3. The method according to claim 1, wherein at least seven biomarkers are determined.

4. The method according to claim 3, wherein the at least 7 biomarkers are MTAP, PTEN, Bax, Bcl-X, β-Catenin, CD20 and Cox-2.

5. A method of preparing a tailored pharmaceutical composition for a patient having a malignant melanoma, the method comprising
(i) determining in melanoma cells comprised in a sample obtained from said malignant melanoma the presence or amount of at least five biomarkers selected from the group comprising or consisting of MTAP, PTEN, Bax, Bcl-X, β-Catenin, CD20, Cox-2, CD49d and MLH1, wherein the absence or decreased amount of β-Catenin and MTAP and/or the presence or increased amount of PTEN, Bax, Bcl-X, CD20, Cox-2, CD49d and MLH1 is associated with a disadvantageous course of disease;
(ii) deriving a treatment regimen for the individual patient based on the presence or amount of biomarkers determined in step (i); and
(iii) providing at least one pharmaceutical compound based on the treatment regimen derived in step (ii).

6. The method according to claim 5, wherein the at least five biomarkers include CD20, Cox-2 and/or MTAP.

7. The method according to claim 5, wherein at least seven biomarkers are determined.

8. The method according to claim 7, wherein the at least 7 biomarkers are MTAP, PTEN, Bax, Bcl-X, β-Catenin, CD20 and Cox-2.

9. The method according to claim 1, wherein the sample comprises melanoma cells.

10. The method according to claim 1, wherein the sample is a tissue sample, a blood sample or a lymph sample.

11. The method according to either of claim 1 or 5, wherein the presence or amount of the biomarkers is analyzed by methods determining genetic or epigenetic modifications or transcriptional or protein levels or a combination thereof.

12. The method according to claim 111, wherein the presence or amount of the biomarkers is determined by immunohistochemistry, mass spectrometry, Western Blot, Northern Blot, PCR, RNA in situ hybridisation or a combination thereof.

13. The method according to claim 12, wherein the biomarkers are protein.

14. A kit for predicting the course of disease in a patient having a malignant melanoma, the kit comprising:
(a) means for determining the presence or amount of the set of biomarkers as defined by any one of claims 1 to 4 in a sample obtained from said malignant melanoma,
(b) instructions how to use the kit.

15. A kit for deriving a treatment regimen for an individual patient having a malignant melanoma, the kit comprising:
(a) means for determining the presence or amount of the set of biomarkers as defined by step (i) of any one of claims 5 to 8 in a sample obtained from said malignant melanoma,
(b) instructions how to use the kit.

16. A pharmaceutical composition for use in treating or preventing malignant melanoma, wherein the pharmaceutical composition comprises (an) inhibitor(s) of CD20, Cox-2 or (an) agent(s) affecting MTAP signalling pathways and/or the oncogenic pathway addiction of PTEN.

17. The method according to claim 1, wherein the sample is obtained from a primary tumour, a lymph node or a metastasis.


SEQUENCE LISTING (22 sequences)

SEQ ID NO: 1 - DNA, 4937 bp, Homo sapiens
ctccgcactgctcactcccgcgcagtgaggttggcacagccaccgctctgtggctcgcttggttcccttagtcccgagcgctcgcccactgcagattcctttcccgtgcagacatggcctctggcaccaccaccaccgccgtgaagattggaataattggtggaacaggcctggatgatccagaaattttagaaggaagaactgaaaaatatgtggatactccatttggcaagccatctgatgccttaattttggggaagataaaaaatgttgattgcgtcctccttgcaaggcatggaaggcagcacaccatcatgccttcaaaggtcaactaccaggcgaacatctgggctttgaaggaagagggctgtacacatgtcatagtgaccacagcttgtggctccttgagggaggagattcagcccggcgatattgtcattattgatcagttcattgacaggaccactatgagacctcagtccttctatgatggaagtcattcttgtgccagaggagtgtgccatattccaatggctgagccgttttgccccaaaacgagagaggttcttatagagactgctaagaagctaggactccggtgccactcaaaggggacaatggtcacaatcgagggacctcgttttagctcccgggcagaaagcttcatgttccgcacctggggggcggatgttatcaacatgaccacagttccagaggtggttcttgctaaggaggctggaatttgttacgcaagtatcgccatggcgacagattatgactgctggaaggagcacgaggaagcagtttcggtggaccgggtcttaaagaccctgaaagaaaacgctaataaagccaaaagcttactgctcactaccatacctcagatagggtccacagaatggtcagaaaccctccataacctgaagaatatggcccagttttctgttttattaccaagacattaaagtagcatggctgcccaggagaaaagaagacattctaattccagtcattttgggaattcctgcttaacttgaaaaaaatatgggaaagacatgcagctttcatgcccttgcctatcaaagagtatgttgtaagaaagacaagacattgtgtgtattagagactcctgaatgatttagacaacttcaaaatacagaagaaaagcaaatgactagtaaacatgtgggaaaaaatattacattttaagggggaaaaaaaaacccaccattctcttctccccctattaaatttgcaacaataaagggtggagggtaatctctactttcctatactgccaaagaatgtgaggaagaaatgggactctttggttatttattgatgcgactgtaaattggtacagtatttctggagggcaatttggtaaaatgcatcaaaagacttaaaaatacggacgtactttgtgctgggaactctacatctagcaatttctctttaaaaccatatcagagatgcatacaaagaattatatataaagaagggtgtttaataatgatagttataataataaataattgaaacaatctgaatcccttgcaattggaggtaaattatgtcttagttataattagattgtgaatcagccaactgaaaatcctttttgcatatttcaatgtcctaaaaagacacggttgctctatatatgaagtgaaaaaaggatatggtagcattttatagtactagttttgctttaaaatgctatgtaaatatacaaaaaaactagaaagaaatatatataaccttgttattgtatttgggggagggatactgggataatttttattttctttgaatctttctgtgtcttcacatttttctacagtgaatttaatcaaatagtaaagttgttgtaaaaataaaagtggatttagaaagatccagttcttgaaaacactgtttctggtaatgaagcagaatttaagttggtaatattaaggtgaatgtcatttaagggagttacatctttattctgctaaagaagaggatcattgatttctgtacagtcagaacagtacttgggtttgcaacagctttctgagaaaagctaggtgtttaatagtttaactgaaagtttaactatttaaaagactaaatgcacattttatggtatctgatattttaaaaagtaatgtttgattctcctttttatgagttaaattattttatacgagttggtaatttttgctttttaataaagtggaagcttgcttttttaactctttttttattgttattttatagaaatgctttttgttggccgggcacagttgctcatccatgtaatcccagcactgtgggaggccgagacgggtggatcacaaggtcaggagatcgagaccatcctggctaatgcgttgaaactccgtctctactaaaaatacaaaaaattagctgggcgtggtggtgggcacctgtagtcccagctactcaggaggctgaggcaggagaatggtgtgaacctgggaggtggagcttgcagtgagcagagcttgcagtgagacgagcttgtgccactgcactccagcctgggcaacagagtaagactcagtctcaaaaaaaaaaaaaagagtgaaatgctttttgtttgcttcagttttttatcatggggagatctttttcctcagaattgttttcttttcactgtaggctattacaggatacttcaggatcaagatacagaaccttttatttaaagagtttgtaaagtcaatgtgtttgtttgtgtctctgagattgacttcaagataataagctgctaattgtaaacaaaacagttaccctccagtattaatatgactcattagtgtgagccatttgggtcaagtatgattatgacccttggacttcctgatgtagtattaaatttcaactctggttatccattagcaatctgtagagaacttaatgaacctgaacccaggcttctctagctctggtaacgtgtgattgttttcactacaatatgatacatagatggtaccttacttttcctcattcttaataggtgtctaagaatgtcagggcaaaagtatgggcatttttcttgctatgttcagaaagtacagttctctccaacttgcagaggtacttttcttgattaaatagccttctctagcaacatcattttcagactaactaaatgaatgcagtatactcttttctttgttctcaatcattcactccttatgcaaagccaatataattttcctcataccttatgcttgaggatattgttgaagaacacttcctggaacacttctcacttgtgatgctgtactaattttttttttttaatttaagctagtatactaagtgaacaccatggtcagttgtgagcattttggtttccgcaaaggatggatggtgagcatcatgggaaagctgtagtttagtgacttagcccttagtgattaatagatttgcatgtacatagaagtctttgttggccttataatctgctgttatatttggcatggattttcatggttttgagaatgacatcctggccctgtggtccccgagggtcatggtccttgtgacctggcccctgttcactgcccccttcgctagcacgagttgctgtgcagggctggaggtagctaccatggcttgtttcaaggaaggaaactctggtacggtggcaccctcaggagtggaggacagtgaacttccttgaagagggagtgactaaggtgacctccaacctgccctgagccagctgccctgcaggtgccacgtgagcctgctctggcatccacaggatgctcctggagcctcttctctggctgctacctcagggcatggttgtggccccaccaacacctattttccaaataattattcattcttgtgacagtggcctgaacatgtttttaattttctcaacaagcatttagccagcacttatccagtgaaacaatttgataaggtttcaaggagtatctgatgggttaggaagtcacgaaatgaggagttcttgccacatttgcagagtccctccttgataaggtttggcggtgtccccacccaaatctcatgttgaattgtagttcccataatccccacatgttgtgggagggacccagtgggaggtaattaaatcatgggggtggttacccccacactgctgttctcatgatactgagttctcacaagtcctgtttgttttataaggggcttttcccccttttgctcaacacttcttcctgccatcatgtgaagaaggacgtgtttgtttccccttctgccacgattgtaagtttcctgaggccttcccagctatgtggaactgtgagttaattaaacctctttcctttataaattacccagtcatgggcagtcctttacagcagcatgagaatggactaatacactcctcaaatgttttgaagattgttgcaccttggaactaccagtgtgcacacaatctggctcaatgtatatattggcccagcaaggcaaagaactgaagttccaggatggaagaacctgtgttctcctcataatagtatagaataattcaagataggcaagaaggacagcagtaaatgaagaccatggaagaaaagaaggaatgccaaagatcgaggaaatctaccaagactagtagggtagtccagaagaagctgtttcagggcctgttgccagctatgcctttgagaacctcgggatcccaaagaatgaggggaatttcttcagaaagacaatctcggcatgcattatttctttgttttgaagattcactcatgttgcatgcatctgtagcttgtgccttttttattgcctagtagtattctgtcatatgcctatcttacaatttgattatctattcacctgttgatgaatgtttgaattttttccatttgaggaattttatgaataaagctgctataagcatgaaaaaaaaaaaaaaaaaa

SEQ ID NO: 2 - protein, 283 aa, Homo sapiens
MASGTTTTAVKIGIIGGTGLDDPEILEGRTEKYVDTPFGKPSDALILGKIKNVDCVLLARHGRQHTIMPSKVNYQANIWALKEEGCTHVIVTTACGSLREEIQPGDIVIIDQFIDRTTMRPQSFYDGSHSCARGVCHIPMAEPFCPKTREVLIETAKKLGLRCHSKGTMVTIEGPRFSSRAESFMFRTWGADVINMTTVPEVVLAKEAGICYASIAMATDYDCWKEHEEAVSVDRVLKTLKENANKAKSLLLTTIPQIGSTEWSETLHNLKNMAQFSVLLPRH

SEQ ID NO: 3 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 4 - protein, 403 aa, Homo sapiens [sequence omitted]

SEQ ID NO: 5 - DNA, 741 bp, Homo sapiens
tcacgtgacccgggcgcgctgcggccgcccgcgcggacccggcgagaggcggcggcgggagcggcggtgatggacgggtccggggagcagcccagaggcggggggcccaccagctctgagcagatcatgaagacaggggcccttttgcttcaggggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgaggcccccagctgccttggactgtgtttttcctccataaattatggcatttttctgggaggggtggggattgggggacgtgggcatttttcttacttttgtaattattggggggtgtggggaagagtggtcttgagggggtaataaacctccttcgggacacaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 143 aa, Homo sapiens
MDGSGEQPRGGGPTSSEQIMKTGALLLQGMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 7 - DNA, 2575 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 8 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 9 - DNA, 3256 bp, Homo sapiens
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 10 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 11 - DNA, 1146 bp, Homo sapiens
cctcaatgacactcatggaggaaatgctgagagaagcattcagatgcatgacacaaggtaagactgccaaaaatcttgttcttgctctcctcattttgttatttgttttatttttaggagttttgagagcaaaatgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcttggcagcaacggagaaaaactctaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcc

SEQ ID NO: 12 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 13 - DNA, 4507 bp, Homo sapiens
gaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataatgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatatcttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactatttaaaaattgacagatcttttctgaagaaaaaaaaaaaaaaa

SEQ ID NO: 14 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 15 - DNA, 4446 bp, Homo sapiens
ggcgcatggctgcggaagcgaggtgcagaccgaggtcccgagggatcgccctccgggaagcggtgatgctgttgttgtacttcggggtgccaaccgggcactcctacaacctggacccggagaatgcactgctgtaccagggcccctccggcacgctgtttggctactcggtggtgctgcacagccacgggtcgaagcgctggctcatcgtgggggctcccactgccagctggctctctaatgcctcagtggtcaatcctggggcgatttacagatgcgggatcagaaagaatccaaaccagacctgcgaacagctccagctgggtagccccagtggagagccttgtgggaagacatgcctggaggagagggataaccagtggctgggggtcaccctttccagacagcctggagaaaatggctctatcgtgacttgtgggcacaggtggaaaaatattttttacatgaagagcgataacaaactccccactggcatttgctacgtcatgccttctgatttgcggacagaactgagtaaaaggatggcgccgtgttgcaaagattatacgagaaaatttggagaaaattttgcatcatgtcaagctggaatatctagtttttacacacaggatttaattgtgatgggggccccgggatcatcgtactggactggcaccgtctttgtctacaatataactacaaaccaatacaaagcatttgtagacagacagaaccaagtaaaatttggaagctacttaggctactcagttggagctggacattttcgaagtccacatactaccgaagtcgtgggaggagcccctcaacacgaacagataggaaaagcatatatatttagcattgatgaaaacgaactgaacatcgtatatgaaatgaaaggtaaaaagcttggctcatactttggagcttctgtctgcgctgtggacctcaatgcagatggcttctcagatctccttgttggagctcccatgcagagcaccatcagggaggaaggaagagtattcgtgtacatcaactctggcatgggagctgtgatggttgaaatggaaagggtccttgtcggaagtgacaaatatgctgcaagatttggggagtctatagcgaatcttggcgacattgacaatgacggctttgaagatattgctattggtgcaccacaagaagacgacttgcgaggtgctgtctacatttacaatggccgagtcgatggaatctcctccacctactcacagagaattgaaggacagcaaatcagcaaatcattaaggatgtttggacaatctatctcaggacaaattgatgcagacaacaatggatatgttgatgtagccattggtgcatttcactctgattctgcagtgttgctaaggacaaggcctgtagtgattgttgaagcatctttaagccatcctgggtctgtaaataggacaaagtttgactgtactgaaaatggacttccatctgtgtgcatgcatcttacactgtgtttctcatataaaggcaaagaggtcccaggctacatcgttttgttttacaatgtgagcttggatgtgcacaggaaggcagagtctccatcaagattttatttcttctctaatgggacttctgacgtgattacaggaagcatacaagtttcaagcagtagagagaaatgtaggacacaccaggcattcatgcggaaagacgtgcgagacatccttacccccattcatgtagaggccacataccaccttgggcatcatgtgatcaccaaacgaaacactgaggaatttccaccactccagccgatccttcagcagaagaaagaaaaagacgttattagaaaaatgataaactttgcaaggttttgtgcctatgaaaattgctctgctgatctccaagtttctgcaaaagttggatttttgaagtaagtatagtttactctgtttttcatcaaaagagcaaaaagggtaaatgcatgtgggttctagcaacaggggatctcaccctcgaacgtcagcatcatttaaaaatttgctaaataataatgttttgctgtgagggactgagtgatgcttatatcactcattttacatgtaaaatgaatgatcaaatatatatatatatatatatgcactatcatcttttaaagtatgagagcatatgagcatccctattattttatgaagtctgctttaagtcttttgttgtaacttagaaggtatttcaggatttaactgtgccttgagatatcattatcacagtcataactatattttaacattttaaaggcctggtaacaagagtttagttggaaaaaataaactttactacacaatggttagtatactttatggctagaaaatgggttatgcaattcaaactgatatgcaaaatactatattttgacaaacttgaaaaagagtaaactaatttgttctaaattcatggagtaagaagaaaatacaatgtgcttagaatagcatctgtcatagaggaagaactaggtaaataggaatgtctactgttactttttaatggaaaaaaactgcaattatgtatcatgtagaggtttcctacattcaatttgctttgcacatctatagacaatgggaaatgaaaaaaggttaaggatacagagtgaccaggagcaggaatgagaaactagattagaaggtgggacataagcaaaccatgcaactatgtagttttatgtcaatttaactcaagaagagcattggggaaaaggaagtgccaatagacaatggctccacaagattggcctacaggcaagcttgtgacgaatttcttcattgactgattgatttgggaaggcttagccagtgtggacaagtgccgtccctgggctggctggtggtcctggattctacaagaaaacaggctgcacaaagtctctttttcatagcctctgcatcagctcctgcctcaaggttcttgtctggagttccctggatgatggactacaagctatagagaaaatgaatcctttcttccccaagttgattttggtcatgatgttttatcacagcaataggaatcttacctaagaaaatggagctctggtacatggctccttacttgaaaatcactctcaatatcacatttagaattccctctacacattagagatggcatagggatgagaaatgtaaattttacaggatgaacaaacattgagagtcattttattcaaggatcttaaaaacagtagaattcttttcagtcaggcaatgttagcatagccatgggtatctatagttcagaggcaaggagccctcccttgactggtaaaggaacttagtgcttacaagtgattcagttggctcactcaggtaattgagtacctgctttgtgtatcagagcatggtgtgactttgtgggtctattagtttgggaatatataattttccatttgagtattttttctaagatatgtaggtttttggtttatcaagagactttgataaaaagtcgttgtgcaggtgcatgtgcaaagagggagatttgcgaagccatagatgaagtgattccctgtgcttgcatttgggttctctgtatcttcctgctcctccttcattgctaatcttcatgagaaatgagatttcaaagtttacgagaaaatgtcactaagaacgatttcaacttattcaaataatagatagaatcattaattcattttttcttgtttcttgcaaagtaattgtctacttagcatttttaatcagaagtgtttttccaattaaatatcaagcctatgattttaaaattagcatcgggccaaacataaggtggtgtttctttaggatgtgggtcctgagtcatgtccaggaaaaatatccacaatgtctattggaaatgtgtatcctcagggcctagcaaacacagaagtggatgatcacagtcagctattggatgggtcacatggcccctaatggaggagctagagaaattacccaaggagctataggaaactgcaaccctataggtggaacaacaatataaactaaccagtacccgggagctcttgtctttaactgcatatgtatcaaaagatggcctagtcggccatcactgcaaagagaggcccattgaacttgcaaactttatatgccccagtacaggggaacgccagggccaaaaagggggagtgggtgggtaggggattgggggggtgggtatgggggacctttgggatagcattgaaaatgtaaatgaggaaaatacctaataaaaaaaaactccacttccaggagctacagagaaggctcagtggttacgagcgcatgtacagttcttgcaggggacccaagttcacttcccaacatccacagtgggccactaagactgatactcatatatctgagtccatgtgggtatttgtgtttctaacacatatatgcaaacaaacccaaaaataaactcctc

SEQ ID NO: 16 - protein, 642 aa, Homo sapiens
RMAAEARCRPRSRGIALREAVMLLLYFGVPTGHSYNLDPENALLYQGPSGTLFGYSVVLHSHGSKRWLIVGAPTASWLSNASVVNPGAIYRCGIRKNPNQTCEQLQLGSPSGEPCGKTCLEERDNQWLGVTLSRQPGENGSIVTCGHRWKNIFYMKSDNKLPTGICYVMPSDLRTELSKRMAPCCKDYTRKFGENFASCQAGISSFYTQDLIVMGAPGSSYWTGTVFVYNITTNQYKAFVDRQNQVKFGSYLGYSVGAGHFRSPHTTEVVGGAPQHEQIGKAYIFSIDENELNIVYEMKGKKLGSYFGASVCAVDLNADGFSDLLVGAPMQSTIREEGRVFVYINSGMGAVMVEMERVLVGSDKYAARFGESIANLGDIDNDGFEDIAIGAPQEDDLRGAVYIYNGRVDGISSTYSQRIEGQQISKSLRMFGQSISGQIDADNNGYVDVAIGAFHSDSAVLLRTRPVVIVEASLSHPGSVNRTKFDCTENGLPSVCMHLTLCFSYKGKEVPGYIVLFYNVSLDVHRKAESPSRFYFFSNGTSDVITGSIQVSSSREKCRTHQAFMRKDVRDILTPIHVEATYHLGHHVITKRNTEEFPPLQPILQQKKEKDVIRKMINFARFCAYENCSADLQVSAKVGFLK

SEQ ID NO: 17 - DNA, 2662 bp, Homo sapiens
gaagagacccagcaacccacagagttgagaaatttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacataa

SEQ ID NO: 18 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 19 - DNA, 1126 bp, Homo sapiens
tggtactgggtggtcctgcaaaatccccaactttgatgtgcgtgaagttgtgaataatatcagacggcttctggatggagaggagcccctacccatgctcccaagttataagaatttcaaaggtactattgaagaactggcttcaaatcagtatgtgattaatggagaagtagctattctggattctacaaccattgaaatctcagagcttcccatccgaacgtggactcagacatataaagaacaggttctagaacccatgttgaatggaactgagaagacaccctctctaataacagactatagggaataccatacagataccactgtgaagtttgtcataaagatgactgaagaaaaactggcagaggcagagagagtcggcctacacaaagtcttcaaactccagagtagtctcacttgcaactctatggtgctttttgaccatgtaggttgcttaaagaaatatgacactgtgttggatattctaagagacttctttgagctcaggcttaaatattatggattaagaaaagaatggcttctaggaatgcttggtgcagaatcttctaaactgaataatcaagctcgctttatattagagaaaatagatggcaaaatagtcattgaaaataaacctaaaaaagaattaattaaagttctgattcagagaggctacgactctgaccctgtgaaagcctggaaagaagctcagcagaaggttccagatgaagaagaaaatgaagaaagtgacactgaaaccagcaccagtgactctgcagccgaggctgggccgaccttcaactaccttctcgacatgcccctgtggtatctgaccaaggagaagaaggatgagctgtgcaaacaaagaaacgagaaggaacaagagctcaacacattaaagcaaaagagtccatcagatctgtggaaggaagatctggctgtttttattgaagaactggaggttgtcgaggccaaggaaaagcaggatgaacaagtgggccttcctggaaaggcggggaaagcaaaggggaagaaagcacagatgtgtgctgacgtcttgccttctcctcggggcaaaagagtcatcccccaagtgactgtggagatgaaagctgaggcagag

SEQ ID NO: 20 - protein, 375 aa, Homo sapiens
GTGWSCKIPNFDVREVVNNIRRLLDGEEPLPMLPSYKNFKGTIEELASNQYVINGEVAILDSTTIEISELPIRTWTQTYKEQVLEPMLNGTEKTPSLITDYREYHTDTTVKFVIKMTEEKLAEAERVGLHKVFKLQSSLTCNSMVLFDHVGCLKKYDTVLDILRDFFELRLKYYGLRKEWLLGMLGAESSKLNNQARFILEKIDGKIVIENKPKKELIKVLIQRGYDSDPVKAWKEAQQKVPDEEENEESDTETSTSDSAAEAGPTFNYLLDMPLWYLTKEKKDELCKQRNEKEQELNTLKQKSPSDLWKEDLAVFIEELEVVEAKEKQDEQVGLPGKAGKAKGKKAQMCADVLPSPRGKRVIPQVTVEMKAEAE

SEQ ID NO: 21 - DNA, 3851 bp, Homo sapiens
ctctcccaaccgcctcgtcgcactcctcaggctgagagcaccgctgcactcgcggccggcgatgcgggaccccggcgcggccgctccgctttcgtccctgggcctctgtgccctggtgctggcgctgctgggcgcactgtccgcgggcgccggggcgcagccgtaccacggagagaagggcatctccgtgccggaccacggcttctgccagcccatctccatcccgctgtgcacggacatcgcctacaaccagaccatcctgcccaacctgctgggccacacgaaccaagaggacgcgggcctcgaggtgcaccagttctacccgctggtgaaggtgcagtgttctcccgaactccgctttttcttatgctccatgtatgcgcccgtgtgcaccgtgctcgatcaggccatcccgccgtgtcgttctctgtgcgagcgcgcccgccagggctgcgaggcgctcatgaacaagttcggcttccagtggcccgagcggctgcgctgcgagaacttcccggtgcacggtgcgggcgagatctgcgtgggccagaacacgtcggacggctccgggggcccaggcggcggccccactgcctaccctaccgcgccctacctgccggacctgcccttcaccgcgctgcccccgggggcctcagatggcagggggcgtcccgccttccccttctcatgcccccgtcagctcaaggtgcccccgtacctgggctaccgcttcctgggtgagcgcgattgtggcgccccgtgcgaaccgggccgtgccaacggcctgatgtactttaaggaggaggagaggcgcttcgcccgcctctgggtgggcgtgtggtccgtgctgtgctgcgcctcgacgctctttaccgttctcacctacctggtggacatgcggcgcttcagctacccagagcggcccatcatcttcctgtcgggctgctacttcatggtggccgtggcgcacgtggccggcttccttctagaggaccgcgccgtgtgcgtggagcgcttctcggacgatggctaccgcacggtggcgcagggcaccaagaaggagggctgcaccatcctcttcatggtgctctacttcttcggcatggccagctccatctggtgggtcattctgtctctcacttggttcctggcggccggcatgaagtggggccacgaggccatcgaggccaactcgcagtacttccacctggccgcgtgggccgtgcccgccgtcaagaccatcactatcctggccatgggccaggtagacggggacctgctgagcggggtgtgctacgttggcctctccagtgtggacgcgctgcggggcttcgtgctggcgcctctgttcgtctacctcttcataggcacgtccttcttgctggccggcttcgtgtccctcttccgtatccgcaccatcatgaaacacgacggcaccaagaccgagaagctggagaagctcatggtgcgcatcggcgtcttcagcgtgctctacacagtgcccgccaccatcgtcctggcctgctacttctacgagcaggccttccgcgagcactgggagcgcacctggctcctgcagacgtgcaagagctatgccgtgccctgcccgcccggccacttcccgcccatgagccccgacttcaccgtcttcatgatcaagtacctgatgaccatgatcgtcggcatcaccactggcttctggatctggtcgggcaagaccctgcagtcgtggcgccgcttctaccacagacttagccacagcagcaagggggagactgcggtatgagccccggcccctccccacctttcccaccccagccctcttgcaagaggagaggcacggtagggaaaagaactgctgggtgggggcctgtttctgtaactttctccccctctactgagaagtgacctggaagtgagaagttctttgcagatttggggcgaggggtgatttggaaaagaagacctgggtggaaagcggtttggatgaaaagatttcaggcaaagacttgcaggaagatgatgataacggcgatgtgaatcgtcaaaggtacgggccagcttgtgcctaatagaaggttgagaccagcagagactgctgtgagtttctcccggctccgaggctgaacggggactgtgagcgatccccctgctgcagggcgagtggcctgtccagacccctgtgaggccccgggaaaggtacagccctgtctgcggtggctgctttgttggaaagagggagggcctcctgcggtgtgcttgtcaagcagtggtcaaaccataatctcttttcactggggccaaactggagcccagatgggttaatttccagggtcagacattacggtctctcctcccctgccccctcccgcctgtttttcctcccgtactgctttcaggtcttgtaaaataagcatttggaagtcttgggaggcctgcctgctagaatcctaatgtgaggatgcaaaagaaatgatgataacattttgagataaggccaaggagacgtggagtaggtatttttgctactttttcattttctggggaaggcaggaggcagaaagacgggtgttttatttggtctaataccctgaaaagaagtgatgacttgttgcttttcaaaacaggaatgcatttttccccttgtctttgttgtaagagacaaaagaggaaacaaaagtgtctccctgtggaaaggcataactgtgacgaaagcaacttttataggcaaagcagcgcaaatctgaggtttcccgttggttgttaatttggttgagataaacattcctttttaaggaaaagtgaagagcagtgtgctgtcacacaccgttaagccagaggttctgacttcgctaaaggaaatgtaagaggttttgttgtctgttttaaataaatttaattcggaacacatgatccaacagactatgttaaaatattcagggaaatctctcccttcatttactttttcttgctataagcctatatttaggtttcttttctatttttttctcccatttggatcctttgaggtaaaaaaacataatgtcttcagcctcataataaaggaaagttaattaaaaaaaaaaagcaaagagccattttgtcctgttttcttggttccatcaatctgtttattaaacatcatccatatgctgaccctgtctctgtgtggttgggttgggaggcgatcagcagataccatagtgaacgaagaggaaggtttgaaccatgggccccatctttaaagaaagtcattaaaagaaggtaaacttcaaagtgattctggagttctttgaaatgtgctggaagacttaaatttattaatcttaaatcatgtactttttttctgtaatagaactcggattcttttgcatgatggggtaaagcttagcagagaatcatgggagctaacctttatcccacctttgacactaccctccaatcttgcaacactatcctgtttctcagaacagtttttaaatgccaatcatagagggtactgtaaagtgtacaagttactttatatatgtaatgttcacttgagtggaactgctttttacattaaagttaaaatcgatcttgtgtttcttcaaccttcaaaactatctcatctgtcagatttttaaaactccaacacaggttttggcatcttttgtgctgtatcttttaagtgcatgtgaaatttgtaaaatagagataagtacagtatgtatattttgtaaatctcccatttttgtaagaaaatatatattgtatttatacatttttactttggatttttgttttgttggctttaaaggtctaccccactttatcacatgtacagatcacaaataaatttttttaaatac

SEQ ID NO: 22 - protein, 574 aa, Homo sapiens
MRDPGAAAPLSSLGLCALVLALLGALSAGAGAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGPGGGPTAYPTAPYLPDLPFTALPPGASDGRGRPAFPFSCPRQLKVPPYLGYRFLGERDCGAPCEPGRANGLMYFKEEERRFARLWVGVWSVLCCASTLFTVLTYLVDMRRFSYPERPIIFLSGCYFMVAVAHVAGFLLEDRAVCVERFSDDGYRTVAQGTKKEGCTILFMVLYFFGMASSIWWVILSLTWFLAAGMKWGHEAIEANSQYFHLAAWAVPAVKTITILAMGQVDGDLLSGVCYVGLSSVDALRGFVLAPLFVYLFIGTSFLLAGFVSLFRIRTIMKHDGTKTEKLEKLMVRIGVFSVLYTVPATIVLACYFYEQAFREHWERTWLLQTCKSYAVPCPPGHFPPMSPDFTVFMIKYLMTMIVGITTGFWIWSGKTLQSWRRFYHRLSHSSKGETAV